SEQUENCE LISTING (22 sequences)

SEQ ID NO: 1 - protein, 770 aa, Homo sapiens
MFPQSRHPTPHQAAGQPFKFTIPESLDRIKEEFQFLQAQYHSLKLECEKLASEKTEMQRHYVMYYEMSYGLNIEMHKQTEIAKRLNTICAQVIPFLSQEHQQQVAQAVERAKQVTMAELNAIIGQQQLQAQHLSHGHGPPVPLTPHPSGLQPPGIPPLGGSAGLLALSSALSGQSHLAIKDDKKHHDAEHHRDREPGTSNSLLVPDSLRGTDKRRNGPEFSNDIKKRKVDDKDSSHYDSDGDKSDDNLVVDVSNEDPSSPRASPAHSPRENGIDKNRLLKKDASSSPASTASSASSTSLKSKEMSLHEKASTPVLKSSTPTPRSDMPTPGTSATPGLRPGLGKPPAIDPLVNQAAAGLRTPLAVPGPYPAPFGMVPHAGMNGELTSPGAAYASLHNMSPQMSAAAAAAAVVAYGRSPMVGFDPPPHMRVPTIPPNLAGIPGGKPAYSFHVTADGQMQPVPFPPDALIGPGIPRHARQINTLNHGEVVCAVTISNPTRHVYTGGKGCVKVWDISHPGNKSPVSQLDCLNRDNYIRSCKLLPDGCTLIVGGEASTLSIWDLAAPTPRIKAELTSSAPACYALAISPDSKVCFSCCSDGNIAVWDLHNQTLVRQFQGHTDGASCIDISNDGTKLWTGGLDNTVRSWDLREGRQLQQHDFTSQIFSLGYCPTGEWLAVGMESSNVEVLHVNKPDKYQLHLHESCVLSLKFAYCGKWFVSTGKDNLLNAWRTPYGASIFQSKESSSVLSCDISVDDKYIVTGSGDKKATVYEVIY

SEQ ID NO: 2 - protein, 743 aa, Homo sapiens
MYPQGRHPTPLQSGQPFKFSILEICDRIKEEFQFLQAQYHSLKLECEKLASEKTEMQRHYVMYYEMSYGLNIEMHKQAEIVKRLSGICAQIIPFLTQEHQQQVLQAVERAKQVTVGELNSLIGQQLQPLSHHAPPVPLTPRPAGLVGGSATGLLALSGALAAQAQLAAAVKEDRAGVEAEGSRVERAPSRSASPSPPESLVEEERPSGPGGGGKQRADEKEPSGPYESDEDKSDYNLVVDEDQPSEPPSPATTPCGKVPICIPARRDLVDSPASLASSLGSPLPRAKELILNDLPASTPASKSCDSSPPQDASTPGPSSASHLCQLAAKPAPSTDSVALRSPLTLSSPFTTSFSLGSHSTLNGDLSVPSSYVSLHLSPQVSSSVVYGRSPVMAFESHPHLRGSSVSSSLPSIPGGKPAYSFHVSADGQMQPVPFPSDALVGAGIPRHARQLHTLAHGEVVCAVTISGSTQHVYTGGKGCVKVWDVGQPGAKTPVAQLDCLNRDNYIRSCKLLPDGRSLIVGGEASTLSIWDLAAPTPRIKAELTSSAPACYALAVSPDAKVCFSCCSDGNIVVWDLQNQTMVRQFQGHTDGASCIDISDYGTRLWTGGLDNTVRCWDLREGRQLQQHDFSSQIFSLGHCPNQDWLAVGMESSNVEILHVRKPEKYQLHLHESCVLSLKFASCGRWFVSTGKDNLLNAWRTPYGASIFQSKESSSVLSCDISRNNKYIVTGSGDKKATVYEVVY

SEQ ID NO: 3 - protein, 767 aa, Homo sapiens
MYPQGRHPAPHQPGQPGFKFTVAESCDRIKDEFQFLQAQYHSLKVEYDKLANEKTEMQRHYVMYYEMSYGLNIEMHKQTEIAKRLNTILAQIMPFLSQEHQQQVAQAVERAKQVTMTELNAIIGQQQLQAQHLSHATHGPPVQLPPHPSGLQPPGIPPVTGSSSGLLALGALGSQAHLTVKDEKNHHELDHRERESSANNSVSPSESLRASEKHRGSADYSMEAKKRKAEEKDSLSRYDSDGDKSDDLVVDVSNEDPATPRVSPAHSPPENGLDKARSLKKDAPTSPASVASSSSTPSSKTKDLGHNDKSSTPGLKSNTPTPRNDAPTPGTSTTPGLRSMPGKPPGMDPIGIMASALRTPISITSSYAAPFAMMSHHEMNGSLTSPGAYAGLHNIPPQMSAAAAAAAAAYGRSPMVGFDPHPPMRATGLPSSLASIPGGKPAYSFHVSADGQMQPVPFPHDALAGPGIPRHARQINTLSHGEVVCAVTISNPTRHVYTGGKGCVKIWDISQPGSKSPISQLDCLNRDNYIRSCKLLPDGRTLIVGGEASTLTIWDLASPTPRIKAELTSSAPACYALAISPDAKVCFSCCSDGNIAVWDLHNQTLVRQFQGHTDGASCIDISHDGTKLWTGGLDNTVRSWDLREGRQLQQHDFTSQIFSLGYCPTGEWLAVGMESSNVEVLHHTKPDKYQLHLHESCVLSLKFAYCGKWFVSTGKDNLLNAWRTPYGASIFQSKESSSVLSCDISADDKYIVTGSGDKKATVYEVIY

SEQ ID NO: 4 - protein, 764 aa, Homo sapiens
MIRDLSKMYPQTRHPAPHQPAQPFKFTISESCDRIKEEFQFLQAQYHSLKLECEKLASEKTEMQRHYVMYYEMSYGLNIEMHKQAEIVKRLNAICAQVIPFLSQEHQQQVVQAVERAKQVTMAELNAIIGQQLQAQHLSHGHGLPVPLTPHPSGLQPPAIPPIGSSAGLLALSSALGGQSHLPIKDEKKHHDNDHQRVSPSASFRGAEKHRNSADYSSESKKQKTEEKEIAARYDSDGEKSDDNLVVDVSNEDPSSPRGSPAHSPRENGLDKTRLLKKDAPISPASIASSSSTPSSKSKELSLNEKSTTPVSKSNTPTPRTDAPTPGSNSTPGLRPVPGKPPGVDPLASSLRTPMAVPCPYPTPFGIVPHAGMNGELTSPGAAYAGLHNISPQMSAAAAAAAAAAAYGRSPVVGFDPHHHMRVPAIPPNLTGIPGGKPAYSFHVSADGQMQPVPFPPDALIGPGIPRHARQINTLNHGEVVCAVTISNPTRHVYTGGKGCVKVWDISHPGNKSPVSQLDCLNRDNYIRSCRLLPDGRTLIVGGEASTLSIWDLAAPTPRIKAELTSSAPACYALAISPDSKVCFSCCSDGNIAVWDLHNQTLVRQFQGHTDGASCIDISNDGTKLWTGGLDNTVRSWDLREGRQLQQHDFTSQIFSLGYCPTGEWLAVGMENSNVEVLHVTKPDKYQLHLHESCVLSLKFAHCGKWFVSTGKDNLLNAWRTPYGASIFQSKESSSVLSCDISVDDKYIVTGSGDKKATVYEVIY

SEQ ID NO: 5 - protein, 264 aa, Homo sapiens
MCHKNGFPQEGGITAAFLQKRKLRLSKNHRPARAKVTEHVRGTRPGRATAGPAASTRAAGSLFFDRWGNRGPAGCRGSSHLPQQLKFTTSDSCDRIKDEFQLLQAQYHSLKLECDKLASEKSEMQRHYVMYYEMSYGLNIEMHKQAEIVKRLNGICAQVLPYLSQEHQQQVLGAIERAKQVTAPELNSIIRQQLQAHQLSQLQALALPLTPLPVGLQPPSLPAVSAGTGLLSLSALGSQAHLSKEDKNGHDGDTHQEDDGEKSD

SEQ ID NO: 6 - DNA, 2313 bp, Homo sapiens
atgttcccgcagagccggcacccgacgccgcaccaggctgcaggccagcccttcaagttcactatcccggagtccctggaccggattaaagaggaattccagttcctgcaggcgcagtatcacagccttaaattggaatgtgagaaactggcaagtgaaaagacagaaatgcagaggcactatgtgatgtattatgaaatgtcatatggattaaacattgaaatgcacaaacagactgaaatcgccaagagattgaatacgatttgtgcacaagtcatcccatttctgtctcaggaacatcaacaacaggtggcccaggctgttgaacgtgccaaacaggtgaccatggcagagttgaatgccatcatcgggcagcagcagttgcaagctcagcatctttctcatggccacggacccccagttccccttacgcctcacccttcgggacttcagcctcctggaatcccgcccctcgggggcagtgccggccttcttgcgctgtctagtgctctgagtgggcagtctcacttggcaataaaagatgacaagaagcaccacgatgcagagcaccacagagacagagagccgggcacaagtaattccctcctggtcccagacagtctaagaggcacagataaacgcagaaatggacctgaattttccaatgacatcaagaaaaggaaggtggatgataaggactccagccactatgacagtgatggtgacaaaagcgatgacaacttagttgtggatgtgtctaatgaggacccttcttctccgcgagcaagccctgcccactcgccccgggaaaatggaatcgacaaaaatcgcctgctaaagaaggatgcttctagcagtccagcttccacggcctcctcggcaagttccacttctttgaaatccaaagaaatgagcttgcatgaaaaagccagcacgcctgttctgaaatccagcacaccaacgcctcggagcgacatgccaacgccgggcaccagcgccactccaggcctccgtccaggtctcggcaagcctccagccatagaccccctcgttaaccaagcggcagctggcttgaggacacccctggcagtgcccggcccatatcctgctccttttgggatggtcccccacgctggcatgaacggcgagctgaccagcccaggcgctgcctacgccagtttacacaacatgtcgccccagatgagcgccgcagccgccgcggccgccgtggtggcctacgggcgctcccccatggtggggtttgatcctccccctcacatgagagtacctaccattcctccaaacctggcaggaatccctggggggaaacctgcatactccttccacgttactgcagacggtcagatgcagcctgtcccttttccccccgacgccctcatcggacccggaatcccccggcatgctcgccagatcaacaccctcaaccacggggaggtggtgtgcgctgtgaccatcagcaaccccacgagacacgtgtacacaggcgggaagggctgcgtcaaggtctgggacatcagccaccctggcaataagagccctgtctcccagctcgactgtctgaacagagacaattatatccgttcctgtaaattgctacccgatggctgcactctcatagtgggaggggaagccagtactttgtccatttgggacctggcggctccaaccccgcgcatcaaggcggagctgacgtcctcggcccccgcctgctacgccctggccatcagccccgattccaaggtctgcttctcatgctgcagcgacggcaacatcgctgtgtgggatctgcacaaccagacactagtgaggcaattccagggccacacagacggagccagctgtattgacatttctaatgatggcaccaagctctggacgggtggtttggacaacacagtcaggtcctgggacctgcgcgaggggcggcagctgcagcagcacgacttcacctcccagatcttctccctggggtactgccccaccggggagtggctggcagtgggcatggagagcagcaatgtggaggtgctgcacgtgaacaagcctgacaagtaccagctgcacctgcatgagagctgcgtgctgtccctgaaatttgcttactgtggtaaatggtttgtgagtactggaaaagataacctcctcaatgcttggcggaccccctatggagccagcatattccagtccaaagagtcctcgtcagtgcttagctgtgacatctctgtggatgataagtacatagtcactggctcgggggacaagaaggctacagtctatgaagtcatctactga

SEQ ID NO: 7 - DNA, 2232 bp, Homo sapiens
atgtacccccagggaaggcacccgaccccgctccagtccggccagcccttcaagttctcgatcttggagatctgcgaccgcatcaaagaagaattccagtttcttcaggctcaataccacagcctcaagctagaatgtgagaagctggccagcgagaagacggaaatgcagcgacattatgtcatgtattatgagatgtcgtacgggctcaacattgaaatgcataagcaggcggagattgtgaagcgtctgagcggtatctgcgctcagattatccccttcctgacccaggagcatcagcagcaggtgctccaggccgtagaacgcgccaagcaggtcaccgtgggggagctgaacagcctcatcgggcagcagctccagccgctgtcccaccacgcaccccctgtgcccctcaccccccgcccagccgggctggtgggcggcagtgctacggggctgcttgctctgtctggagccctggctgcccaggctcagctggcggcggctgtcaaggaggaccgtgcgggcgtggaggccgaggggtccagagtggagagagccccgagcaggagtgcatctccctcgccccctgagagtctcgtggaggaggagcgaccgagtggccctggtggtggcgggaagcagagagcagatgagaaggagccatcaggaccttatgaaagcgacgaagacaagagtgattacaatctggtggtggacgaggaccaaccctcagagccccccagcccggctaccaccccctgcggaaaggtacccatctgcattcctgcccgtcgggacctggtggacagtccagcctccttggcctctagccttggctcaccgctgcctagagccaaggagctcatcctgaatgaccttcccgccagcactcctgcctccaaatcctgtgactcctccccgccccaggacgcttccacccccgggcccagctcggccagtcacctctgccagcttgctgccaagccagcaccttccacggacagcgtcgccctgaggagccccctgactctgtccagtcccttcaccacgtccttcagcctgggctcccacagcactctcaacggagacctctccgtgcccagctcctacgtcagcctccacctgtccccccaggtcagcagctctgtggtgtacggacgctcccccgtgatggcatttgagtctcatccccatctccgagggtcatccgtctcttcctccctacccagcatccctgggggaaagccggcctactccttccacgtgtctgcggacgggcagatgcagccggttcccttcccctcggatgcactggtaggcgcgggcatcccgcggcacgcccggcagctgcacacgctggcccatggcgaggtggtctgcgcggtcaccatcagcggctccacacagcatgtgtacacgggcggcaagggctgtgtgaaggtgtgggacgtgggccagcctggggccaagacgcccgtggcccagctcgactgcctgaaccgagacaactacattcgttcctgcaagttgctgccggatggccggagtctgatcgtgggcggtgaggccagcaccttgtccatttgggacctggcggcgcccaccccccgtatcaaggccgagctgacttcctcagccccagcctgctacgccctggccgtcagccccgacgccaaggtttgcttctcctgctgcagcgatggcaacattgtggtctgggacctgcagaatcagactatggtcaggcagttccagggccacacggacggcgccagctgcattgatatttccgattacggcactcggctctggacagggggcctggacaacacggtgcgctgctgggacctgcgggagggccgccagctgcagcagcatgacttcagctcccagattttctccctgggccactgccctaaccaggactggctggcggtcggaatggagagtagcaacgtggagatcctgcacgtccgcaagccggagaaataccagctgcacctccacgagagctgcgtgctgtccctgaagtttgcctcctgcggacggtggtttgtgagcaccgggaaggacaacctgctcaacgcctggaggacgccgtacggggccagcattttccagtccaaggagtcgtcctcagtcctgagttgtgacatctccagaaataacaaatacatcgtgacaggctcgggggacaagaaggccaccgtgtatgaggtggtctactga

SEQ ID NO: 8 - DNA, 2319 bp, Homo sapiens
atgtatccgcagggcagacatccggctccccatcaacccgggcagccgggatttaaattcacggtggctgagtcttgtgacaggatcaaagacgaattccagttcctgcaagctcagtatcacagcctcaaagtggagtacgacaagctggcaaacgagaagacggagatgcagcgccattatgtgatgtactatgagatgtcctatggcttgaacattgaaatgcacaagcagacagagattgcgaagagactgaacacaattttagcacagatcatgcctttcctgtcacaagagcaccagcagcaggtggcgcaggcagtggagcgcgccaagcaggtcaccatgacggagctgaacgccatcatcgggcagcagcagctccaggcgcagcacctctcccatgccacacacggccccccggtccagttgccaccccacccgtcaggtctccagcctccaggaatccccccagtgacagggagcagctccgggctgctggcactgggcgccctgggcagccaggcccatctgacggtgaaggatgagaagaaccaccatgaactcgatcacagagagagagaatccagtgcgaataactctgtgtcaccctcggaaagcctccgggccagtgagaagcaccggggctctgcggactacagcatggaagccaagaagcggaaggtggaggagaaggacagcttgagccgatacgacagtgatggagacaagagtgatgatctggtggtggatgtttccaatgaggaccccgcaacgccccgggtcagcccggcacactcccctcctgaaaatgggctggacaaggcccgtagcctgaaaaaagatgcccccaccagccctgcctcggtggcctcttccagtagcacaccttcctccaagaccaaagaccttggtcataacgacaaatcctccacccctgggctcaagtccaacacaccaaccccaaggaacgacgccccaactccaggcaccagcacgaccccagggctcaggtcgatgccgggtaaacctccgggcatggacccgataggtataatggcctcggctctgcgcacgcccatctccatcaccagctcctatgcggcgcccttcgccatgatgagccaccatgagatgaacggctccctcaccagtcctggcgcctacgccggcctccacaacatcccaccccagatgagcgccgccgccgctgctgcagccgctgcctatggccgatcgccaatggtgagctttggagctgttggttttgaccctcaccccccgatgcgggccacaggcctcccctcaagcctggcctccattcctggaggaaaaccagcgtactcattccatgtgagtgctgatgggcagatgcagcccgtgcccttcccccacgacgccctggcaggccccggcatcccgaggcacgcccggcagatcaacacactcagccacgggggggtggtgtgtgccgtgaccatcagcaaccccagcaggcacgtctacacaggtggcaagggctgcgtgaagatctgggacatcagccagccaggcagcaagagccccatctcccagctggactgcctgaacagggacaattacatgcgctcctgcaagctgcaccctgatgggcgcacgctcatcgtgggcggcgagggcagcacgctcaccatctgggacctggcctcgcccacgccccgcatcaaggccgagctgacgtcctcggctcccgcctgttatgccctggccattagccctgacgccaaagtctgcttctcctgctgcagcgatgggaacattgctgtctgggacctgcacaaccagaccctggtcaggcagttccagggccacacagatggggccagctgcatagacatctcccatgatggcaccaaactgtggacagggggcctggacaacacggtgcgctcctgggacctgcgggagggccgacagctacagcagcatgacttcacttcccagatcttctcgctgggctactgccccactggggagtggctggctgtgggcatggagagcagcaacgtggaggtgctgcaccacaccaagcctcacaagtaccagctgcacctgcacgagagctgcgtgctctccctcaagttcgcctactgcggcaagtggttcgtgagcactgggaaagataaccttctcaacgcctggaggacgccttatggagccagcatatcccagtctaaagaatcctcgtctgtcttgagttgtgacatttcagcggatgacaaatacattgtaacaggctctggtgacaagaaggccacagtttatgaggtcatctactaa

SEQ ID NO: 9 - DNA, 2322 bp, Homo sapiens
atgattcgcgacctgagcaagatgtacccgcagaccagacacccggcaccgcatcagcctgctcaaccctttaaatttacaatttccgaatcctgtgatcggattaaggaagagtttcagtttttacaggctcaataccacagtctgaagctggaatgtgagaaactcgccagtgagaagacagagatgcagcggcattatgtcatgtattatgaaatgtcctatgggttgaatatagaaatgcacaagcaggcagagattgtcaagaggctgaatgctatctgtgcacaagtcattcctttcctgtcccaagagcaccagcaacaagtggtgcaggctgtggaacgggccaagcaggtgaccatggcagaactgaacgccatcattgggcaacaactccaggcccagcatttatcacatggacatggtctccccgtacctctgactccacacccttcagggctccagccccctgccattccacccatcggtagcagtgccgggcttctggccctctccagtgctctaggaggtcagtcccatcttccaattaaagatgagaagaagcaccatgacaatgatcaccaaagagacagagactccatcaagagctcttcagtatccccatcagccagtttccgaggtgctgagaagcacagaaactccgcagactactcctcagagagcaaaaagcagaaaactgaagaaaaggaaattgcagctcgttatgacagcgatggtgagaaaagtgatgacaacttggtggttgacgtttccaatgaggatccatcttcccctcgagggagcccagcacattcccccagagagaatggcctagacaagacacgcctgctcaagaaagatgccccgattagtccagcctctattgcatcttccagcagtactccctcctccaaatccaaagaacttagccttaatgaaaaatctactactcccgtctcaaagtccaatacccctactccacgaactgatgcgcccaccccaggcagtaactctactcccggattgaggcctgtacctggaaaaccaccaggagttgaccctttggcctcaagcctaaggaccccaatggcagtaccttgtccatatccaactccatttgggattgtgccccatgctggaatgaacggagagctgaccagccccggagcggcctacgctgggctccacaacatctcccctcagatgagcgcagctgctgccgccgccgctgctgctgctgcctatgggagatcaccagtggtgggatttgatccacaccatcacatgcgtgtgccagcaatacctccaaacctgacaggcattccaggaggaaaaccagcatactccttccatgttagcgcagatggtcagatgcagcctgtcccttttccacccgacgccctcatcggacctggaatcccccggcatgctcgccagatcaacaccctcaaccacggggaggtggtgtgcgcggtgaccatcagcaaccccacgagacacgtgtacacgggtgggaagggctgcgtcaaggtctgggacatcagccacccaggcaataagagtcctgtctcccagctcgactgtctgaacagggataactacatccgttcctgcagattgctccctgatggtcgcaccctaattgttggaggggaagccagtactttgtccatttgggacctggcggctccaaccccacgcatcaaggcagagctgacatcctcggcccccgcctgctatgccctggccatcagccccgattccaaggtctgcttctcatgctgcagcgacggcaacatcgctgtgtgggatctgcacaaccagaccttggtgaggcaattccagggccacacagatggagccagctgtattgacatttctaatgatggcaccaagctctggacaggtggtttggacaacacggtcaggtcctgggacctgcgcgaggggcggcagctgcagcagcacgacttcacctcccagatcttttctctgggctactgcccaactggagagtggcttgcagtggggatggagaacagcaatgtggaagttttgcatgtcaccaagccagacaaataccaactacatcttcatgagagctgtgtgctgtcgctcaagtttgcccattgtggcaaatggtttgtaagcactggaaaggacaaccttctgaatgcctggagaacaccttatggggccagtatattccagtccaaagaatcctcatcggtgcttagctgtgacatctccgtggacgacaaatacattgtcactggctctggggataagaaggccacagtttatgaagttatttattaa

SEQ ID NO: 10 - DNA, 795 bp, Homo sapiens
atgtgtcacaagaatggcttccctcaggaaggcggcattaccgctgcctttctgcagaaaaggaaactaaggctcagcaagaaccaccgcccagccagagccaaggtcacagagcacgtccgtggcacgcgtccaggtcgtgccacagcagggccggcggcttcgacgcgggcagccgggtcccttttctttgacagatggggaaaccgaggcccggccggctgccggggctcctcgcacctaccccagcaactcaaattcaccacctcggactcctgcgaccgcatcaaagacgaatttcagctactgcaagctcagtaccacagcctcaagctcgaatgtgacaagttggccagtgagaagtcagagatgcagcgtcactatgtgatgtactacgagatgtcctacggcttgaacatcgagatgcacaaacaggctgagatcgtcaaaaggctgaacgggatttgtgcccaggtcctgccctacctctcccaagagcaccagcagcaggtcttgggagccattgagagggccaagcaggtcaccgctcccgagctgaactctatcatccgacagcagctccaagcccaccagctgtcccagctgcaggccctggccctgcccttgaccccactacccgtggggctgcagccgccttcgctgccggcggtcagcgcaggcaccggcctcctctcgctgtccgcgctgggttcccaggcccacctctccaaggaagacaagaacgggcacgatggtgacacccaccaggaggatgatggcgagaagtcggattag

SEQ ID NO: 11 - protein, 780 aa, Mus musculus
MFPQSRHPTPHQAAGQPFKFTIPESLDRIKEEFQFLQAQYHSLKLECEKLASEKTEMQRHYVMYYEMSYGLNIEMHKQTEIAKRLNTICAQVIPFLSQEHQQQVAQAVERAKQVTMAELNAIIGVRGLPGLPPTQQQLQAQHLSHGHGPPVPLTPHPSGLQPPGIPPLGGSASLLALSSALSGQSHLAIKDDKKHHDAERHRDREPGTSNSLLVPDSLRGTDKRRNGPEFSSDIKKRKVDDKDNYDSDGDKSDDNLVVDVSNEDPSSPHASPTHSPRENGIDKNRLLKKDASGSPASTASSGSSSSLKSKEVSLHEKANTPVLKSSTPTPRSDMPTPGTSATPGLRPGLGKPPAMEPLVNQAAAGLRTPLAVPGPYPAPFGMVPHAGMNGELTSPGAAYAGLHSMSPQMSAAAAAAAAAVVAYGRSPMVGFDPPPHMRVPSIPPNLAGIPGGKPAYSFHVTADGQMQPVPFPPDALIGPGIPRHARQINTLNHGEVVCAVTISNPTRHVYTGGKGCVKVWDISHPGNKSPVSQLDCLNRDNYIRSCKLLPDGCTLIVGGEASTLSIWDLAAPTPRIKAELTSSAPACYALAISPDSKVCFSCCSDGNIAVWDLHNQTLVRQFQGHTDGASCIDISNDGTKLWTGGLDNTVRSWDLREGRQLQQHDFTSQIFSLGYCPTGEWLAVGMESSNVEVLHVNKPDKYQLHLHESCVLSLKFAYCGKWFVSTGKDNLLNAWRTPYGASIFQSKESSSVLSCDISVDDKYIVTGSGDKKATVYEVIY

SEQ ID NO: 12 - protein, 767 aa, Mus musculus
MYPQGRHPTPLQSGQPFKFSVLEICDRIKEEFQFLQAQYHSLKLECEKLASEKTEMQRHYVMAAPHQCPQGGTSYPHWPRLSPLQYYEMSYGLNIEMHKQAEIVKRLSAICAQMVPFLTQEHQQQVLQAVDRAKQVTVGELNSLLGQQNQLQPLSHAPPVPLTPRPAGLVGAGATGLLALSGALAAQAQLVAAVKEDRVGVDAEGSRVDRAASRSSSPSPPESLVEEDHPSSRGGSGKQQRAEDKDLSGPYDSEEDKSDYNLVVDEDQPSEPPSPVTTPCGKAPLCIPARRDLTDSPASLASSLGSPLPRSKDIALNDLPTGTPASRSCGTSPPQDSSTPGPSSASHLCQLAAQPAAPTDSIALRSPLTLSSPFTSSFSLGSHSTLNGDLSMPGSYVGLHLSPQVSSSVVYGRSPLQMAFESHPHLRGSSVSLPGIPVAKPAYSFHVSADGQMQPVPFPSDALVGTGIPRHARQLHTLAHGEVVCAVTISSSTQHVYTGGKGCVKVWDVGQPGSKTPVAQLDCLNRDNYIRSCKLLPDGQSLIVGGEASTLSIWDLAAPTPRIKAELTSSAPACYALAVSPDAKVCFSCCSDGNIVVWDLQNQAMVRQFQGHTDGASCIDISDYGTRLWTGGLDNTVRCWDLREGRQLQQHDFSSQIFSLGHCPNQDWLAVGMESSHVEVLHVRKPEKYQLRLHESCVLSLKFASCGRWFVSTGKDNLLNAWRTPYGASIFQSKESSSVLSCDISRNNKYIVTGSGDKKATVYEVVY

SEQ ID NO: 13 - protein, 782 aa, Mus musculus
MYPQGRHPAPHQPGQPGFKFTVAESCDRIKDEFQFLQAQYHSLKVEYDKLANEKTEMQRHYVMYYEMSYGLNIEMHKQTEIAKRLNTILAQIMPFLSQEHQQQVAQAVERAKQVTMTELNAIIGVRGLPNLPLTQQQLQAQHLSHATHGPPVQLPPHPSGLQPPGIPPVTGSSSGLLALGALGSQAHLAVKDEKNHHELDHRERESSTNNSVSPSESLRASEKHRGSADYSMEAKKRKAEEKDSLSRYDSDGDKSDDLVVDVSNEDPATPRVSPAHSPPENGLDKARGLKKDAPTSPASVASSSSTPSSKTKDLGHNDKSSTPGLKSNTPTPRNDAPTPGTSTTPGLRSMPGKPPGMDPIGIMASALRTPITLTSSYPAPFAMMSHHEMNGSLTSPSAYAGLHNIPSQMSAAAAAAAAAYGRSPMVSFGAVGFDPHPPMRATGLPSSLASIPGGKPAYSFHVSADGQMQPVPFPHDALAGPGIPRHARQINTLSHGEVVCAVTISNPTRHVYTGGKGCVKIWDISQPGSKSPISQLDCLNRDNYIRSCKLLPDGRTLIVGGEASTLTIWDLASPTPRIKAELTSSAPACYALAISPDAKVCFSCCSDGNIAVWDLHNQTLVRQFQGHTDGASCIDISHDGTKLWTGGLDNTVRSWDLREGRQLQQHDFTSQIFSLGYCPTGEWLAVGMESSNVEVLHHTKPDKYQLHLHESCVLSLKFAYCGKWFVSTGKDNLLNAWRTPYGASIFQSKESSSVLSCDISADDKYIVTGSGDKKATVYEVIY

SEQ ID NO: 14 - protein, 773 aa, Mus musculus
MIRDLSKMYPQTRHPAPHQPAQPFKFTISESCDRIKEEFQFLQAQYHSLKLECEKLASEKTEMQRHYVMYYEMSYGLNIEMHKQAEIVKRLNAICAQVIPFLSQEHQQQVVQAVERAKQVTMAELNAIIGQQLQAQHLSHGHGLPVPLTPHPSGLQPPAIPPIGSSAGLLALSSALGGQSHLPIKDEKKHHDNDHQRDRDSIKSSSVSPSASFRGSEKHRNSTDYSSESKKQKTEEKEIAARYDSDGEKSDDNLVVDVSNEDPSSPRGSPAHSPRENGLDKTRLLKKDAPISPASVASSSSTPSSKSKELSLNEKSTTPVSKSNTPTPRTDAPTPGSNSTPGLRPVPGKPPGVDPLASSLRTPMAVPCPYPTPFGIVPHAGMNGELTSPGAAYAGLHNISPQMSAAAAAAAAAAAYGRSPVVGFDPHHHMRVPAIPPNLTGIPGGKPAYSFHVSADGQMQPVPFPPDALIGPGIPRHARQINTLNHGEVVCAVTISNPTRHVYTGGKGCVKVWDISHPGNKSPVSQLDCLNRDNYIRSCRLLPDGRTLIVGGEASTLSIWDLAAPTPRIKAELTSSAPACYALAISPDSKVCFSCCSDGNIAVWDLHNQTLVRQFQGHTDGASCIDISNDGTKLWTGGLDNTVRSWDLREGRQLQQHDFTSQIFSLGYCPTGEWLAVGMENSNVEVLHVTKPDKYQLHLHESCVLSLKFAHCGKWFVSTGKDNLLNAWRTPYGASIFQSKESSSVLSCDISVDDKYIVTGSGDKKATVYEVIY

SEQ ID NO: 15 - protein, 197 aa, Mus musculus
MMFPQSRHSGSSHLPQQLKFTTSDSCDRIKDEFQLLQAQYHSLKLECDKLASEKSEMQRHYVMYYEMSYGLNIEMHKQAEIVKRLNGICAQVLPYLSQEHQQQVLGAIERAKQVTAPELNSIIRQQLQAHQLSQLQALALPLTPLPVGLQPPSLPAVSAGTGLLSLSALGSQTHLSKEDKNGHDGDTHQEDDGEKSD

SEQ ID NO: 16 - protein, 497 aa, Homo sapiens
MVRSRQMCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 17 - DNA, 1494 bp, Homo sapiens
atggtgaggagcaggcaaatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctag

SEQ ID NO: 18 - DNA, 2313 bp, Mus musculus
atgttcccgcagagccggcacccaacsccgcaccaagctgcaggccagccctttaagttcactatcccggagtctctggaccggattaaagaggaattccagttcctgcaggcgcagtatcacagtcttaaattggagtgtgagaaactggcaagtgaaaagacagaaatgcagaggcactacgtgatgtattatgaaatgtcatatggattaaacattgaaatgcacaaacagactgaaatcgccaagagattgaacaccatttgtgcccaagacatcccatttctgtctcaggaacatcaacaacaggtggcccaggctgtggaacgtgccaaacaggtgaccatggcagagttgaatgccatcatcgggcagcagcagttgcaagctcagcatctctcccatggccatggacccccagtacctctcacgcctcacccttcaggacttcagcctcctggaatcccgcccctcgggggcagtgccggccttcttgcgctgtctagtgctctgagtgggcagtctcacttggcaataaaagatgacaagaagcaccatgatgcagagcgccacagagacagagagcctggcacgagtaattccctcttggtcccagacagcctaagaggcacagataagcgcagaaatggtccagagttttccagtgacatcaaaaaaaggaaggtggatgataaggataactatgacagtgatggggacaagagtgatgacaacttagttgtggatgtgtctaacgaggacccttcttctccacatgcaagccccacacactcaccccgggaaaacggaattgacaaaaaccgtctgctgaagaaagatgcttcaggtagcccggcatccacagcctcctctggaagttcctcttccctgaaatccaaagaagtgagcctgcatgaaaaagccaacactcctgttctgaaatccagcacaccgacgcctcggagcgacatgccaaccccaggcaccagcgctactccaggcctccgtccaggtcttggcaagcctccagccatggaaccccttgtcaaccaagcagcagctggcctgaggacgcccctggcagtgcctggcccataccctgccccctttggcatggtgccccatgcgggcatgaacggagagctgaccagccctggtgctgcctatgcaggtctacacagcatgtctccacagatgagcgctgcagctgctgcagctgctgctgctgtggtggcctatgggcgctccccaatggttggttttgatcctcctcctcacatgagagtaccttctatcccccccaacctggcaggaatacctggagggaaaccagcatactccttccacgttactgctgatggccaaatgcagcctgttccttttccccctatgcccctcattggacctggattcccccgacatgctcggcagatcaacaccctcaaccacggggaggtggtgtgcgcagtgaccatcagcaaccccacaaggcacgtgtacacaggtggcaagggctgcgttaaggtgtgggacatcagccaccccggcaacaagagccccgtctctcagctggattgtctgaatagagataactacatccgaacctgtaaattgctacctgatgactgtactctcatagtgggaggggaagccagtactttgtccatttgggacctggcggctccaaccccgcgcatcaaggcggagctgacgtcctcggcccccgcctgctacgccctggccatcagccccgactccaaggtctgcttctcatgctgcagtgacggcaacatcgcagtgtgggacctgcacaaccagaccctggtgaggcaattccagggccacacagacggagccagctgtattgacatttctaatgatggcaccaagctctggacaggcggtttagacaacactgtgaggtcctgggacctgagagaagggcggcagctgcagcagcatgacttcacttcacagatcttctctttgggatactgcccaactggggagtggcttgctgtgggcatggaaagcagcaacgtggaagttctgcatgtgaacaagcctgacaagtaccagctgcacctccacgagagctgcgtgctgtccctaaagtttgcttattgtggcaaatggtttgtgagtactggaaaagataacctcctcaatgcttggcggaccccctatggagccagcatattccagtccaaagagtcctcgtcagtgcttagctgtgacatctctgtggatgataagtacatagtcactggctcgggggacaagaaggctacagtctatgaagtcatctactga

SEQ ID NO: 19 - DNA, 2304 bp, Mus musculus
atgtaccctcagggaaggcacccgaccccgctgcagtctggccagcctttcaagttctcagtactggaaatctgtgaccggatcaaagaggaattccagtttcttcaagctcagtaccacagcctcaagctagaatgtgagaagctggccagcgagaagacagaaatgcaaaggcattatgtgatggctgcaccccatcagtgtccccagggtggcaccagctatccacactggccaagactgtctcctttgcagtactacgagatgtcctacggactcaacattgagatgcataaacaggctgagattgtgaaacgcctcagtgcgatctgtgcccagatggtcccgttcctcactcaggagcatcagcagcaggtgctccaggctgtggaccgagccaagcaggtgaccgtgggggaactgaacagcctcctggggcagcagaatcagctccagccgctgtcccacgcaccccccgtgcctctcaccccgcgcccagccggcctggtgggtgccggggccactgggctgctggccctatctggggcactggctgcgcaggcccagctggtggctgccgtaaaggaagaccgtgtgggtgtggacgccgaggggtccagagtggacagagctgccagcaggagttcttccccgtctccccctgagagtctggtggaagaggaccatcccagcagccgaggcggcagtgggaaacagcagagagctgaagacaaggatctgtcagggccttatgacagtgaggaagacaagagtgactataacctggtagtggatgaggaccaaccgtcagagccccccagccctgtgaccaccccttgtgggaaggcgcccctctgcattcctgcccgcagggacctcacagacagtccagcctccttggcctccagtttgggctcaccactccccagaagcaaagacatagccctgaacgatcttcccacaggcactcctgcctccaggtcatgtggtacctctccaccccaggactcgtccaccccggggcccagctcagccagtcacctctgccagctggcggctcagccggcagcacccacagacagcatcgccctgaggagtcccctgaccttgtccagccctttcacctcatccttcagcctgggctcccacagcaccctcaatggggacctctccatgcctggctcctatgtcggcctccacctgtccccccaggtcagcagctctgtcgtgtatggacgctcacctctgcagatggcatttgaatcgcacccccatctccgaggctcgtctgtctccttgcctggcatccctgtggctaagccggcttactccttccacgtgtctgcggatggccagatgcagcctgtgcccttcccgtctgatgctctggtaggcacaggcatccctcgccacgcaaggcagctacacacgctggcccacggtgaggtggtgtgtgccgtcaccatcagcagctccacacagcacgtgtacacaggcggcaaaggatgcgtgaaggtgtgggacgtgggccagccgggtagcaagacccctgtggcacagctggattgcctgaaccgagacaactacatccgctcctgcaagctgctgcccgacgggcagagcctgattgtaggtggcgaggccagtaccctgtccatttgggacctggcagcacccacaccacggatcaaagcagagctgacttcgtctgccccggcctgctatgctctggccgtcagtccggacgccaaggtctgcttctcctgctgcagcgacggtaacatcgtggtctgggacctgcagaaccaggccatggtcagacagttccagggccacacggacggggccagctgcatcgacatatcagactacgggacccggctgtggactgggggcctggacaacactgtgcgctgctgggacctgcgcgagggccgccagcttcagcagcatgacttcagttcccagattttctcattgggccactgtcccaatcaggactggttggctgtggggatggagagcagccacgtggaggtcctgcatgtgcgcaagcctgagaagtaccagctccgcctccatgagagctgcgtgctgtcactcaagttcgcttcctgtggacgctggtttgtgagcacaggcaaggacaacctgctcaatgcctggaggacaccctatggggccagcatttttcagtccaaagaatcatcttctgtactgagctgcgacatctccaggaataataagtacatcgtgacaggctcaggggacaagaaggccactgtgtatgaggtggtgtactga

SEQ ID NO: 20 - DNA, 2349 bp, Mus musculus
atgtatccgcaaggcagacatccggcaccccatcaacccgggcagccgggatttaaattcactgtggccgagtcctgtgacaggatcaaagacgaattccagttcctgcaagctcagtatcacagcctcaaagtggagtatgacaagctggctaacgagaagacggagatgcagcgccattatgtgatgtactatgagatgtcctatggcttgaatattgaaatgcacaagcagacagagattgcgaagagactgaacacaatcctagcccagatcatgccttttttgtcacaggagcatcagcagcaagtggcgcaggctgtggaacgcgccaagcaggtcaccatgacggagttgaacgccatcatcggggtacgtggactccccaatctgcctctcacccagcagcaactccaggcccagcacctctcccatgccacgcatggtcccccggtccagctgccaccccacccgtcaggcctccagcctcctgggattcccccagtgacaggaagcagctctgggttgctggcacttggtgccctgggaagtcaagctcacttggcggtgaaggatgagaagaaccaccatgaactggatcacagagagagagagtccagcacgaacaattccgtgtcaccctctgaaagcctccgggccagtgagaagcaccggggctctgcagactacagcatggaagccaagaagcggaaggcggaagagaaagacagcctcagcagatacgatagcgatggggacaagagtgacgacctggtggtggatgtctctaatgaggacccagcaacaccccgggtgagcccagcacactcccctcctgaaaatgggctggacaaagcccgtggtctgaagaaagatgcccccaccagcccagcctccgtggcttcctccagcagcacaccttcctccaagaccaaagaccttggtcataatgacaaatcttccacacctgggctcaagtccaacacaccaacgccaagaaatgatgccccaactccaggcaccagcaccaccccggggctccggtcaatgccgggcaaacctccaggcatggacccgataggtataatggcctcggccctgcgaacacccatcaccctcaccagctcctatccagcaccctttgccatgatgagccaccacgagatgaatggctccctcaccagcccaagcgcctatgctggcctacacaacatcccatcccagatgagcgccgccgcagccgctgcagccgccgcctatggccgatcgccaatggtgagctttggagctgttggttttgaccctcaccccccaatgagggccacaggcctgccttccagtctcgcctccattcctggagggaaaccggcatactccttccatgtgagtgctgatgggcagatgcaacctgtgcccttcccccatgatgcactagcaggccctggcattcccaggcatgcccggcagatcaatacgctcagccatggagaggtggtatgtgctgtgaccatcagcaaccccacacgacacgtctacacaggcggcaagggctgtgtgaagatatgggacatcagccagccgggcagcaagagtcccatctcccagctggactgcctgaacagggacaactacatccgctcgtgcaagcttctccccgatgggcgcacgctcattgtgggtggtgaggccagcacgctcaccatctgggacctggcctcacccacaccccgcatcaaggctgagctgacgtcctcggctccagcctgttatgccctggccatcagtcctgatgccaaagtctgtttttcctgctgcagcgacgggaacattgcggtttgggatctgcacaaccagaccctggtcaggcagttccagggccacacagatggggccagctgtatagacatctctcatgatggcactaagctgtggaccgggggcctggacaacaccgtgcgctcctgggacctacgtgaaggacggcagttacagcaacacgatttcacctcccagatcttctccctgggttactgccccactggggagtggctggccgtgggcatggagagcagcaatgtggaggtcctgcaccacactaagcccgacaaataccagctgcacctgcacgagagctgcgtgctgtccctcaagttcgcctattgtggcaagtggtttgtgagcactgggaaagacaaccttctcaatgcctggaggacgccttatggagccagcatcttccagtcaaaagaatcctcatctgtcttgagctgtgacatttcagcggatgacaaatatattgtaacaggctctggtgacaagaaggccacagtttacgaggtcatctactga

SEQ ID NO: 21 - DNA, 2322 bp, Mus musculus
atgattcgcgacctgagcaagatgtacccgcagacgcgccacccggcaccgcatcagcctgctcaacccttcaaatttacaatttcagaatcctgtgatcggattaaggaagagtttcagtttttacaggctcaataccacagtctgaagctggaatgtgagaagctcgccagcgagaagacagagatgcagcggcattatgtcatgtattatgaaatgtcctatgggttgaacatagaaatgcacaagcaggcagagattgttaaacgactaaatgctatctgtgcacaggtcattcctttcctgtcccaagagcaccagcaacaagtggtgcaggctgtggaacgggccaagcaggtgaccatggcagaactgaacgccatcattgggcaacaactccaagctcagcatttatcacatggacatggtctgcctgtgcctctgacaccacacccttcagggcttcagcccccagccatcccacctatcggtagcagtgcaggacttctggccctctccagtgcactaggaggtcagtctcacctcccaattaaggatgagaagaagcaccatgacaatgatcaccaaagagacagagactccatcaagagctcttccgtatccccatcagccagtttccggggttctgagaaacataggaactctacagactattcctcagagagcaaaaagcagaaaacagaagaaaaagaaattgcagctcgctatgacagtgatggtgagaagagtgatgacaacttggtagttgatgtgtccaatgaggatccatcttccccccgaggaagcccagcacattccccaagggagaatggcctggacaagacacgactcctcaagaaagatgcccccatcagccccgcttctgttgcatcttccagcagtactccttcctccaaatccaaagagcttagccttaatgaaaagtctactactcctgtttcaaagtccaatacccctactccacgaactgatgcacctacccctggcagtaattctactcctggactgaggcctgtacctggaaagccaccaggtgtggaccccttagcatcaagcctgaggaccccgatggctgtaccttgtccatatccaaccccctttgggatcgtgccccatgctgggatgaacggagagctgaccagccccggagccgcctatgctgggctacacaacatctccccccagatgagtgcagctgctgctgcagctgcggcagcagcagcctatggaagatcacctgtggtgggatttgatccacaccatcacatgcgagtgccagcaatacctccaaatctaacaggcattccaggaggaaaaccagcatactcctttcacgtcagtgccgatggccagatgcagcctgtccctttcccccctgatgccctcattggaccagggatcccccgacacgctcgacagatcaacaccctcaaccatggggaggtggtgtgtgcagtgaccatcagcaaccccacaaggcacgtgtacacaggtggcaagggctgtgttaaggtctgggacatcagccaccctggcaacaagagcccagtctctcagctggactgtctgaacagggataactacatccgttcctgcagattgctccctgatggtcgcaccttaattgttggaggggaagccagcacactgtccatctgggacctggcagctccaactccacgcatcaaggcagagctgacatcctcagcccctgcctgctatgctctggccatcagccccgactccaaggtctgcttctcatgctgcagcgacggtaacatcgcagtgtgggatctgcacaaccagactctggtgaggcaattccaggggcacacagatggagccagctgtattgacatttctaatgatggcaccaagctctggacaggtggtttggacaacactgtgaggtcctgggacctgcgtgaagggcggcagctgcagcaacatgacttcacctctcagatcttttcattgggctattgcccaactggagagtggcttgcagtggggatggagaatagcaatgtggaagtattgcatgtcaccaaaccagacaaataccagttgcatcttcatgagagctgtgtgctgtcactcaagtttgcccactgtggcaaatggtttgtaagcactggaaaggacaaccttctgaatgcttggaggacgccttatggggccagcatattccagtccaaagaatcctcatcggtgcttagctgtgacatctctgtggatgacaagtacattgtcactggctctggggacaagaaagctacggtttatgaagttatttattaa

SEQ ID NO: 22 - DNA, 594 bp, Mus musculus
atgatgtttccgcaaagccggcactcgggctcctcccacctccctcagcagctcaagttcaccacctccgactcctgtgaccgcatcaaagatgagttccagctgctgcaagcgcagtatcacagcctgaagctggagtgcgacaagctggccagcgagaagtcagagatgcagaggcattacgtcatgtactatgagatgtcctacggattgaacatcgagatgcacaaacaggcggaaattgtgaagaggctgaatgggatttgcgcccaggttctgccctatttgtcacaggagcatcagcagcaggtcctgggagccatcgagagagccaagcaggtcacggctcctgagctgaactccatcatccgacagcagctccaggctcaccagctgtcccagctgcaggcgctggccctgcccctgacaccgctgcctgtcgggctccagccaccgtccctccctgcagtcagtgcaggcacaggcctgctgtcactctctgctctgggttctcagacccacctctccaaggaggacaagaacggacacgatggggacacccaccaggaggatgacggagagaagtcggattag

(19) United States
(12) Patent Application Publication
(10) Pub. No.: US 2014/0315732 A1
(43) Pub. Date: Oct. 23, 2014

(54) CANCER DIAGNOSIS AND TREATMENT

(71) Applicant: Corrinne Lobe, Toronto (CA)

(72) Inventor: Corrinne Lobe, Toronto (CA)

(73) Assignee: Miami Mice Research Corp, Toronto, ON (CA)

(21) Appl. No.: 14/000,386

(22) PCT Filed: Feb. 23, 2012
(86) PCT No.: PCT/CA2012/000161

§ 371 Date: Oct. 29, 2013

Related U.S. Application Data
(60) Provisional application No. 61/445,972, filed on Feb. 23, 2011.

(51) Int. Cl. G01N 33/68

(52) U.S. Cl. CPC G01N 33/6893
USPC 506/9; 506/18

(57) ABSTRACT

A diagnostic assay for cancer such as lung cancer is disclosed. The assay can also be used to follow patients during treatment and for assessment of disease relapse after treatment. The assay utilizes biomarkers for tumor formation identified in a transgenic mouse model. The assay is used to identify a therapeutic indication for a patient based on the patient's biomarker expression. The biomarker and fragments thereof are also useful for treating cancer, for example lung or colon cancer.

### CROSS-REFERENCE TO RELATED APPLICATIONS

This application claims the benefit of U.S. 61/445,972 filed Feb. 23, 2011, which is incorporated herein by reference in its entirety.

### TECHNICAL FIELD

This invention relates to methods for the diagnosis of cancer using biological markers and to treatment of cancer.

### BACKGROUND

Lung cancer has been the most common cancer for several decades and causes the largest number of cancer deaths in the world. In 2008, there were an estimated 1.61 million new cases in the world (12.7% of the total) and 1.38 million deaths (18.2% of the total) caused by cancer of the lung. This exceeds the death rates of breast, prostate and colorectal cancer combined. Lung cancer affects smokers, former smokers and non-smokers, the latter group comprising approximately 15% of cases. The basis for tumor progression and aggressive biological behavior of this disease remains poorly understood. Compounding the problem is the paucity of available animal models, making it difficult to determine the biological and molecular origins of the disease and optimal treatment strategies.

As with other cancers, the survival rate for lung cancer is much higher if it is detected early. When the disease is detected in an early, localized stage and can be removed surgically, the five-year survival rate can reach 85%. But once the cancer has spread to other organs, especially to distant sites, as few as 2% of patients survive five years. Unfortunately, lung cancer is usually asymptomatic until it has reached an advanced stage. Thus, only 5% of lung cancers are found at an early, localized stage. There is, therefore, a compelling need for tools that aid in the screening of asymptomatic persons leading to detection of lung cancer in its earliest, most treatable stages.

Potential screening tools to detect early stage lung cancer are chest X-ray and computed tomography (CT) scanning. However, the high cost and high rate of false positives render these radiographic tools impractical for routine widespread use. For example, a recent study of the U.S. National Cancer Institute concluded that screening for lung cancer with chest X-rays can detect early lung cancer but produces many false-positive test results, causing needless follow-up testing, Oken et al., Journal of the National Cancer Institute, 97(24)1832-1839, 2005. A similar problem with false positives is encountered with ongoing trials involving CT scans. Specificity of CT screening is calculated at around 65% based on the number of indeterminate radiographic findings. The large number of indeterminate pulmonary nodules found on prevalence CT scanning require further investigation by invasive procedures and many of the pulmonary nodules identified by CT scanning are benign, which raises serious concerns about the large portion of incurred health care costs per life saved.

PET scans are another diagnostic option, but PET scans are costly and generally not amenable for use in screening programs.

Currently, age and smoking history are the only two risk factors that have been used as selection criteria by the large screening studies. A blood test that could detect radiographically apparent cancers (>0.5 cm) as well as occult and pre-malignant cancer (below the limit of radiographic detection) would identify individuals for whom radiologic screening is most warranted and de facto would reduce the number of benign pulmonary findings that require further workup.

It is clear, therefore, that there is an urgent need for improved lung cancer screening and better detection tools that overcome the aforementioned limitations of radiographic techniques.

In addition to a diagnostic screen for lung cancer, it would be of great value if the detection tools could provide an indication of the optimal therapeutic treatments. For cancers such as breast and colon cancer, targeted therapies for patients with mutations in epidermal growth factor receptor (EGFR) or GTPase KRas (KRAS) have shown success in clinical trials. This approach of targeted or “personalized medicine” can improve patient response and also save health care costs because treatments can be tailored specifically to a patient.

A few methods for diagnosis of cancer using biological markers and the use of biological markers for therapeutic indication have been described. However, none of these are useful for cost-effective, minimally invasive, highly predictive analytical means that aid in the screening of asymptomatic persons leading to detection of lung cancer in its earliest, most treatable stages. Technical problems associated with identifying markers of cancer have been unsatisfactorily addressed in the prior art by using human serum samples in prospective studies. In the prospective studies, patients at high risk of developing lung cancer were screened by CT scan, samples were taken to be banked for testing, and the patients were followed for several years to determine which patients develop lung cancer. When it is known which patients develop lung cancer, the banked samples from those patients can be tested for marked differences in DNA or protein biomarkers. The major problem with using the results of these prior art studies for detecting cancer in patients suspected of having cancer or for screening patients at risk is that this approach identifies biomarkers for mid to late stage lung cancer while what is needed are biomarkers that provide early detection of lung cancer using minimally invasive, and cost effective methods, and that have the potential as biomarkers to be targeted therapeutically and to be used as prognostic indicators and indicators of a patient's response to therapy.

### SUMMARY OF INVENTION

One technical problem that was overcome in order to aid in the development of the invention described herein is that biomarkers for lung cancer are difficult to identify in human patients because of the variability in gene and protein expression that is unrelated to the presence of lung cancer. Therefore a suitable lung cancer animal model could provide reproducible and predictable disease development and suitable negative controls.

Another technical problem that was overcome in order to aid in the development of the invention, was to identify specific biomarkers that are quantitatively different in patients with cancer than in patients without cancer. Such biomarkers are very useful in the clinical setting in providing a yes/no answer to a physician rather than requiring statistical analysis of test results, which are not amenable to a simple clinical diagnostic test. Furthermore, in the present invention a threshold expression level can be identified for each biomarker such that assay results indicating a patient's expression level above or below (depending on the chosen biomarker(s)) of the protein can provide a yes or no determination regarding the need for additional screening such as a CT scan. This is an advantage over other methods, which use complex statistical analyses of expression patterns of many proteins/mRNA to compute the likelihood that a patient has cancer.

Another technical problem that was overcome in order to aid in the development of the invention, was to identify biomarkers with known function that can be targeted in cancer treatment; a cancer treatment that can be tested in the animal model. A further technical problem was to determine whether an oncogene in one tissue is necessarily an oncogene in a different tissue.

The applicant has discovered that these technical problems are solved by way of methods that include identifying cancer biomarkers using a lung cancer mouse model, using the biomarkers to identify patients that are likely to have lung cancer, and using a biomarker as a therapeutic indicator to predict the treatment to which the patient is likely to respond. The biomarkers or derivatives of the biomarkers according to the invention described herein may be used as a treatment for cancer. It has also been discovered that valuable and unexpected results may be achieved when the biomarkers are used as anti-cancer agents for tumors of tissue origin other than lung tissue.

In one aspect, the invention is directed to the use of a transgenic mouse model for lung cancer (Grg1 mice) to identify biomarkers of cancer and therapies useful for treating cancer. In one embodiment of this aspect of the invention, Grg1 mice may be used to identify biomarkers that can be quantitatively measured in patient blood samples and detect differences between lung cancer patients and normal control samples.

In another aspect, the invention is directed to assays, methods, and kits for the early detection of lung cancer using tissue or body fluid samples of the patient in which the presence or absence of cancer is to be determined. In one embodiment, the invention relates to detection of lung cancer by evaluating the presence of one or more biomarker, which can be presented as a panel of biomarkers. In one embodiment, the present invention may be employed in a lung cancer screening strategy especially when used in concert with radiographic imaging and other screening modalities of a population of patients, such as patients at risk for cancer. The present invention can be used to enrich the population most likely to have cancer for further radiographic analysis of these patients to rule out the presence of lung cancer. In short, in this aspect, the invention is directed to a method of detecting the presence of lung cancer in a patient.

In one embodiment, a blood sample from the patient is analyzed for the presence of one or a panel of protein biomarkers associated with lung cancer according to the invention. In a particular embodiment, the invention is directed to a screening test using the biomarkers described below for asymptomatic patients, or patients of a high-risk group which have not yet been diagnosed with lung cancer using acceptable tests and protocols, that is, for example, these patients lack radiographically detectable lung cancer. The method of the invention described herein is relatively inexpensive, minimally invasive with high specificity for cancer and provides an alternative to the high cost and low specificity of current lung cancer screening methods, such as chest X-ray or Low Dose CT.

In another aspect, the invention is directed to determining therapeutic interventions, by using the status of one or more biomarkers described below to predict the patient response to one or more drug treatments. In another embodiment of the invention, the biomarkers or analogs, homologs or fragments of the biomarkers may be used to treat cancers.

Thus, according to this aspect of the present invention, a method is provided for the identification of one or more lung tumor protein biomarkers in a biological sample. Such biological samples include body fluids or tissues such as but not limited to blood, serum or plasma or cells (such as those obtained from biopsy). The method comprises the steps of measuring the identified lung tumor protein biomarker expression using, for example, Western blots, proteomic analysis, Northern blots, RT-PCR, immunoassays as described below and identified in the Grg1 mouse model, between lung tumor tissue of individuals identified as not having lung cancer; and measurement of the same lung tumor protein biomarkers in biological samples, such as human blood samples from suspected lung cancer patients, thereby identifying protein biomarkers that are measurably different in lung cancer patients compared to patients that do not have lung cancer (normal or negative controls).

In another aspect of the invention, the invention is directed to a kit containing one or more biomarkers for the detection of cancer, for example, but not limited to, lung cancer. In one embodiment of the invention, the one or more biomarkers pertain to a Groucho-related protein, such as but not limited to TLE1. The kit can include antibodies affixed to a solid support for measuring expression levels of biomarkers, including, but not limited to, Grouch-related protein or proteins in a patient's body fluid such as blood, serum or plasma. The solid support, such as an antibody array, can further include a reference, control or baseline amount of the same one or more biomarkers from samples that are not indicative of cancer for use in determining the likelihood of the presence of cancer in the patient sample.

In various embodiments, the biomarker comprises one or more of a Groucho-related protein, Mdm2, Ras, ErbB1, ErbB2 or CyclinD1/D2. To determine the likelihood of cancer in a patient, in another embodiment of the method of the invention, the method comprises determining a quantitative measure of the amount of one or more of Mdm2, Ras, ErbB1, ErbB2 or CyclinD1/D2 and/or a Groucho-related protein such as but not limited to TLE. In another aspect, the invention is directed to a method for indicating the use of a histone-deacetylase inhibitor (HDAC inhibitor) as a method of treatment in a cancer patient. In one embodiment of this method, a blood sample is taken from the cancer patient and the expression of a Groucho-related protein in the cancer patient is measured and compared to reference values of the expression of this protein from patients that do not have cancer. From this data, the usefulness of treating the patient with a histone-deacetylase inhibitor is determined. In yet another aspect, the invention is directed to a method for evaluating drugs for cancer treatment by applying one or more candidate chemotherapeutic drugs to the Grg1 lung cancer mouse model and quantitatively comparing the size, number, and histological appearance of lung tumors in treated and untreated Grg1 mice.

In still another aspect, the invention is directed to a method for treating cancers. In one embodiment, the method uses derivatives such as analogs, homologs or fragments of the protein biomarkers. For example, in one embodiment, the Q-domain of the Groucho-related proteins can be used to treat lung cancer. In another embodiment, a Groucho-related protein or analogs, homologs or fragments thereof can be used to treat colon cancer.

In another aspect of the invention, the invention is directed to a method for diagnosing lung cancer in a mammal by providing a biological sample from the mammal; providing a panel comprising one or more macromolecules, wherein each macromolecule is a biomarker or specifically binds to a biomarker that is measurable at a quantitatively different level in Grg1-expressing transgenic mice than in non-transgenic mice; contacting the mammalian biological sample to the panel to determine the level of expression of the biomarker; comparing the levels of the biomarker expressed in the mammalian sample to the levels of the biomarker in a reference sample; wherein the presence of a quantitatively different level of expression of the biomarker in the mammalian biological sample compared to the reference sample is indicative of cancer.

In various embodiments, the reference sample is a biological sample from a healthy mammal not diagnosed with cancer and not having increased risk factors for cancer; or a purified biomarker provided at a concentration level corresponding to the level measured in a biological sample from a healthy mammal not diagnosed with cancer and not having increased risk factors for cancer.

In various embodiments, the macromolecule is antibodies, nucleic acids, proteins or fragments thereof.

In various embodiments, the biomarker is proteins, mRNA or antibodies.

In various embodiments, the biomarker is a protein that is a Groucho-related protein, Mdm2, Ras, ErbB1, ErbB2 and CyclinD1/D2; or an mRNA encoding a one such protein; or an autoantibody which specifically binds to one such protein. In a preferred embodiment, at least one of the biomarkers comprises a Groucho-related protein. The biomarker proteins are characterized as proteins that are normally non-secretory proteins.

In various embodiments, the panel includes one or more antibody, wherein the antibody specifically binds to a biomarker protein that is measurable at a quantitatively different level in Grg1-expressing transgenic mice than in non-transgenic mice; or the panel includes one or more cDNA molecules, wherein the cDNA molecules specifically bind to a biomarker mRNA or its complementary sequence, wherein the biomarker mRNA or its complementary sequence is measurable at a quantitatively different level in Grg1-expressing transgenic mice than in non-transgenic mice; or the panel includes one or more protein that is a biomarker that is measurable at a quantitatively different level in Grg1-expressing transgenic mice than in non-transgenic mice.

In some embodiments, the method further includes isolating mRNA from the mammalian biological sample and the reference sample and quantitatively amplifying the mRNA and producing cDNA.

In various embodiments, the mammalian biological sample is a fluid sample. The fluid sample can be blood, plasma or serum. In various other embodiments, the mammalian biological sample and the reference sample are detectably labeled.

In another aspect of the invention, the invention is directed to a method for identifying a candidate patient responsive to inhibitor chemotherapy by providing a biological sample from the candidate patient; detecting the presence of expression of a TLE biomarker in the patient biological sample; comparing the level of expression of TLE biomarker in the patient biological sample to the levels of the TLE biomarker in a reference sample; and identifying the candidate patient as a responsive candidate for HDAC inhibitor chemotherapy when the TLE biomarker expression in the candidate patient biological sample is elevated above the level of TLE biomarker in the reference sample. In one embodiment, the cancer patient has lung cancer. In various embodiments, the expression of TLE biomarker is determined by measuring TLE protein level or mRNA level. The TLE biomarker proteins are characterized as proteins that are normally non-secretory proteins.

In various embodiments, the reference sample is a biological sample from a healthy mammal not diagnosed with cancer and not having increased risk factors for cancer; a purified biomarker provided at a concentration level corresponding to the level measured in a biological sample from a healthy mammal not diagnosed with cancer and not having increased risk factors for cancer, a biological sample from a known non-responsive patient; or a purified biomarker provided at a concentration level corresponding to the level measured in a biological sample from a known non-responsive patient.

In various embodiments, the patient biological sample is a fluid sample or a tissue sample. The fluid sample can be blood, plasma or serum. The tissue sample can be lung tissue.

In another aspect of the invention, the invention is directed to a method for monitoring effectiveness of a cancer patient treatment protocol by providing a biological sample from the patient before undergoing the treatment protocol; detecting the presence of expression of a TLE biomarker in the pre-treatment patient sample; comparing the level of expression of TLE biomarker in the pre-treatment patient sample to the level of expression of TLE biomarker in a biological sample from the patient during or after the treatment, or to the levels of the TLE biomarker in a reference sample, wherein a change in the level of expression of TLE biomarker in the pre-treatment patient sample compared to the post-treatment patient sample, or to a difference in the level of TLE expression of the post-treatment patient sample compared to the level of TLE biomarker in the reference sample is indicative of the effectiveness of the treatment. In one embodiment, a decreased level of TLE in said post-treatment patient sample is indicative of effective treatment. In various embodiments, the expression of TLE biomarker is determined by measuring TLE protein level or mRNA level.

In various embodiments, the reference sample is a biological sample from a healthy mammal not diagnosed with cancer and not having increased risk factors for cancer; or a purified biomarker provided at a concentration level corresponding to the level measured in a biological sample from a healthy mammal not diagnosed with cancer and not having increased risk factors for cancer.

In one embodiment, the treatment includes administering a therapeutically effective amount of a HDAC inhibitor. In one embodiment, the cancer patient has lung cancer.

In various embodiments, the sample is a fluid sample or a tissue sample. The fluid sample can be blood, plasma or serum. The tissue sample can be lung tissue.

In yet another aspect of the invention, the invention is directed to a method for monitoring effectiveness of a cancer patient treatment protocol by providing a post-treatment biological sample from said patient; detecting the presence of expression of a TLE biomarker in said patient sample; comparing the level of expression of TLE biomarker in said post-treatment patient sample to the level of expression of TLE biomarker in a reference sample, wherein a difference in the level of TLE expression of said post-treatment patient sample compared to said level of TLE expression in said reference sample is indicative of the effectiveness of said treatment. In one embodiment, a decreased level of TLE in said post-treatment patient sample is indicative of effective treatment. The TLE biomarker proteins are characterized as proteins that are normally non-secretory proteins.

In one embodiment the reference sample is obtained from a post-treatment cancer patient non-responsive to therapy. In various other embodiments, the reference sample is a biological sample from a healthy mammal not diagnosed with cancer and not having increased risk factors for cancer; or a purified biomarker provided at a concentration level corresponding to the level measured in a biological sample from a healthy mammal not diagnosed with cancer and not having increased risk factors for cancer.

In another aspect of the invention, the invention is directed to a method for identifying a cancer patient treatment candidate by providing a biological sample from the patient; detecting the presence of a higher level of TLE biomarker in the patient biological sample compared to the levels of the TLE biomarker in a reference sample; and identifying the cancer patient as a candidate for treating the cancer with the administration of a therapeutically effective amount of a fragment of a Groucho-related protein. In one embodiment, the cancer patient has lung cancer. The TLE biomarker proteins are characterized as proteins that are normally non-secretory proteins. In one embodiment, the fragment comprises a Q domain.

In various embodiments, the reference sample is a biological sample from a healthy mammal not diagnosed with cancer and not having increased risk factors for cancer; a purified biomarker provided at a concentration level corresponding to the level measured in a biological sample from a healthy mammal not diagnosed with cancer and not having increased risk factors for cancer; a biological sample from a known non-responsive patient; or a purified biomarker provided at a concentration level corresponding the level measured in a biological sample from a known non-responsive patient.

In another aspect of the invention, the invention is directed to a method for identifying a cancer patient treatment candidate by providing a biological sample from the patient; detecting the presence of an inactivating APC or a beta-catenin mutation in the patient biological sample; and identifying the cancer patient having the inactivating APC or the beta-catenin mutation as a candidate for treating the cancer with administration of a therapeutically effective amount of a Groucho-related protein or a fragment of a Groucho-related protein to the cancer patient. In one embodiment, the cancer patient has colon cancer. In one embodiment, the fragment comprises at least a WD40 domain.

In another aspect of the invention, the invention is directed to a method for treating cancer in a mammal by administering a therapeutically effective amount of a Groucho-related protein or a fragment of a Groucho-related protein to the mammal. In one embodiment, the cancer is lung cancer. In one embodiment, the fragment comprises a Q domain. In yet another embodiment, the cancer is colon cancer. In one embodiment, the fragment comprises at least a WD40 domain.

In another aspect of the invention, the invention is directed to a method for treating cancer in a mammal by administering a therapeutically effective amount of a beta-catenin to the cancer. In one embodiment, the cancer is lung cancer.

In another aspect of the invention, the invention is directed to a transgenic mouse whose genome comprises a heterozygous, null allele of the gene encoding APC protein, wherein the Apc gene is truncated at least at codon 1638, and a hemizygous transgene allele of a Groucho-related gene, and wherein the mouse exhibits formation of tumors.

In another aspect of the invention, the invention is directed to a method of screening a compound for anti-tumor activity, the method comprising the steps of preparing a transgenic mouse whose genome comprises a heterozygous, null allele of the gene encoding APC protein, wherein the Apc gene is truncated at least at codon 1638, and a hemizygous transgene allele of a Groucho-related gene, and wherein the mouse exhibits formation of tumors; treating the prepared transgenic mouse with a candidate compound; determining a level of the tumor in the transgenic mouse treated with the candidate compound by measuring the number of tumor cells, volume of the tumor, or tumor cell viability; and identifying the candidate compound as a compound having anti-tumor activity if the number of the tumor cells or the volume of the tumor has been decreased relative to the number of tumor cells or volume of the tumor in the transgenic mouse prior to the treatment with the candidate compound, or if apoptosis of the tumor cells has been induced after the treatment with the candidate compound.

In another aspect of the invention, the invention is directed to a diagnostic kit comprising one or more biomarker that is at a quantitatively different level in Grg1-expressing transgenic mice than in non-transgenic mice.

In one embodiment the one or more biomarker of the kit is a Groucho-related protein, Mdm2, Ras, ErbB1, ErbB2 or CyclinD1/D2. In a preferred embodiment, at least one biomarker comprises a Groucho-related protein. In one embodiment, the kit includes the one or more biomarker provided as a panel of biomarkers bound to a solid support. The biomarker proteins are characterized as proteins that are normally non-secretory proteins.

In another aspect of the invention, the invention is directed to a method for identifying markers for non-small cell lung cancer by providing a biological sample from a Grg1-expressing transgenic mouse and a biological sample from a non-Grg1-expressing mouse; measuring the expression of biomarkers in the Grg1-expressing transgenic mouse and the non-Grg1 expressing mouse; and identifying biomarkers that are expressed at a different level in the samples. The biomarker proteins are characterized as proteins that are normally non-secretory proteins.

In one embodiment, the biomarker is proteins or mRNA. In various embodiments, the samples are a fluid sample or a tissue sample. The fluid sample can be blood, serum or plasma. The tissue sample can be lung tissue.

In one embodiment, the method further includes contacting the biological samples with a binding partner prior to the measuring step. The binding partner can be an antibody, a nucleic acid, a ligand, or an aptamer. In various embodiments, the step of identifying comprises detecting binding to a binding partner. Accordingly, the biological samples can be detectably labeled. In other embodiments, the step of detecting includes contacting the marker-binding partner complex with a second binding partner. In such embodiments, the second binding partner can be detectably labeled. Some embodiments include liquid chromatography analysis and tandem mass spectrometry analysis.

In one embodiment, the level of biomarker expression is increased in the Grg1-expressing transgenic mouse as compared to the level of biomarker expression in the non-Grg1-expressing mouse. In another embodiment, the level of biomarker expression is decreased in the Grg1-expressing transgenic mouse as compared to the level of biomarker expression in the non-Grg1-expressing mouse.

In some embodiments, the method further includes contacting the samples with DNA prior to the step of comparing the mRNA levels. In yet other embodiments, the method further includes isolating mRNA from the samples and quantitatively amplifying the mRNA and producing cDNA.

These and other features, aspects, and advantages of the present invention will become better understood with regard to the following description and appended claims.

### BRIEF DESCRIPTION OF THE DRAWINGS

In the accompanying drawings

FIG. 1 is a diagrammatic representation of a summary of changes in protein expression for the Grg1 and Grg5 transgenic mice;

FIG. 2 is a table of antibody dilutions used for the antibody microarray;

FIG. 3 is a table depicting an Antibody array map;

FIG. 4 is a table showing the detection and spot analysis for the antibody microarray;

FIG. 5 is an example image for the antibody microarray (Negative Image, Array incubated with 145145A5);

FIG. 6 is tables showing sample repartition during slide incubation (14 arrays/slide);

FIG. 7 is a table of the mean values, SD and CV of labeled BSA positive controls for each array or sample;

FIG. 8 is a table of the mean values (net signal, background signal removed), SD and CV of controls: Anti-Albumin and anti-H1 antibodies with each sample;

FIG. 9 is a table of the mean values, SD and CV of negative controls (PBS) and background (B635) for each sample;

FIG. 10 is a table of mean net signal for the serums and for the incubation controls;

FIG. 11 is a table showing an example of highlighted values;

FIG. 12 is the table of results obtained for the 10 antibodies;

FIG. 13 is the Box plots of MDM2 antibody (p=0.03, Maim & Whitney);

FIG. 14 is the Box plots of TLE1 antibody (p=0.03, Mann & Whitney);

FIG. 15 is a table showing the tumor development in the Grg1 transgenic mice after treatment with TrichostatinA;

FIG. 16 is photographs of sections of tumors from the Grg1hPLAP transgenic mice, without and with TrichostatinA treatment;

FIG. 17 is a table showing the quantitation of Intestinal Polyps and Lung Adenomas in APC^{min}/Grg1hPLAP mice; and

FIGS. 18 A and B are schematics showing the structural and sequence conservation of the vertebrate groucho-related proteins.

### DETAILED DESCRIPTION

It is difficult to identify biomarkers using human samples because of the heterogeneity of the samples. Many differences occur among human samples that are not related to a disease. To overcome this difficulty, an animal model that provides predictable disease progression and the availability of suitable negative controls was used in developing the invention disclosed herein.

The Grg1 transgenic mouse line was used to identify biological protein markers for lung cancer. The Grg1 mouse develops lung tumors that resemble human non-small cell lung adenocarcinoma. The Grg1 mice were used as a source of tissue to provide samples for measurement of gene and protein differences during lung tumor development. The production of the Grg1 transgenic mouse is described in Allen, T. et al. “Grg1 Acts as a Lung-Specific Oncogene in a Transgenic Mouse Model”, Cancer Res, 66:3, 1294-1301 (2006) (incorporated herein by reference). In the present invention, the Grg1 transgenic mice were used to identify biological protein markers for lung cancer. The identified biological protein markers relevant for identifying patients with lung cancer were proteins measurable in serum from human cancer patients that were quantitatively or qualitatively different from proteins identified in human control patients that did not have cancer when analyzed by conventional means of diagnosing cancer in humans. The present invention provides an assay and method for detecting lung cancer. In short, a sample, such as a blood sample, from the patient or subject suspected of having cancer or in need of ruling out cancer as a diagnosis is obtained and is analyzed for the presence or absence of biomarkers for lung cancer. One biomarker or a panel of biomarkers is used, each biomarker associated to some degree with lung cancer. The biomarkers according to the invention include but are not limited to Groucho-related proteins, called transducin-like enhancer-of-split (TLE) in humans, auto-antibodies to Groucho-related proteins, or nucleic acids encoding Groucho-related proteins. The biomarkers according to the invention further include, but are not limited to Mdm2 proteins, auto-antibodies to Mdm2 proteins, or nucleic acids encoding Mdm2 proteins.

Groucho proteins are transcriptional co-repressors that interact with a number of transcription factors and histone deacetylase-1 (HDAC-1) to repress transcription of target genes. The Groucho family of proteins is encoded by Grg1-5 in mouse and TLE1-5 in man. The majority of Groucho proteins possess all the domains of the prototype Drosophila Groucho protein, but Grg5/TLE5 and an alternatively spliced variant of Grg3 encode a Groucho isoform with only the amino-terminal Q and G/P domains. The Q domain is used for oligomerization of Groucho proteins. Therefore, the short Groucho proteins may inhibit activity of the long proteins by forming non-functional complexes.

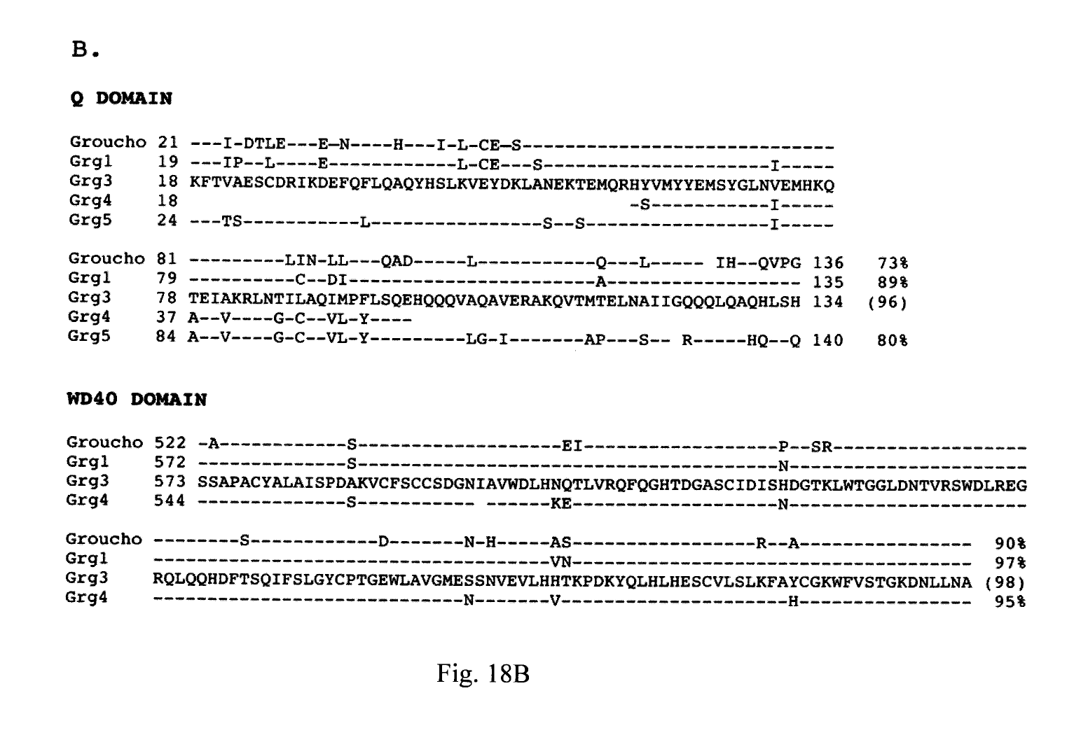

FIG. 18A shows the structural organization of the Drosophila Groucho protein at the top and the related vertebrate proteins, with all of the Groucho domains conserved or containing only the Q and B/P domains, are shown underneath. Nomenclature of the Groucho homologues is indicated in the left columns. The numbers indicate percent amino acid identity with the Droxophila Groucho protein (left of slash) or the human TLE protein (right of slash). FIG. 18B shows an alignment of the Q and WD40 domains of the murine Groucho homologues. Dashes indicate amino acid identity. Percentages at the right indicate homology to Grg3. For Grg3, the conservation to human TLE3 is given in parentheses.

Table 1 identifies the Q domain and WD40 domain by amino acid numbers for each of the TLE proteins. The amino acid and polynucleotide sequences of Grg1-5 and TLE1-5 are provided in Appendix A.

TABLE 1

| Protein | Q domain | WD40 domain |
|---|---|---|
| TLE1 | 1-135 | 483-726 |
| TLE2 | 1-132 | 456-740 |
| TLE3 | 1-124 | 473-764 |
| TLE4 | 8-140 | 477-720 |
| TLE5 | 76-190 | n/a |

The human homologue of Mdm2 (murine double minute 2 (mdm2) gene) encodes an E3 ubiquitin-protein ligase, which is a negative regulator of the p53 tumor suppressor. Overexpression of this gene can result in excessive inactivation of tumor protein p53, diminishing its tumor suppressor function. The amino acid and polynucleotide sequences of human MDM2 are provided in Appendix A.

The biomarkers of the invention, Groucho-related proteins (Grg family of proteins in mice; TLE family of proteins in humans). Mdm2, Ras, ErbB1, ErbB2 and CyclinD1/D2 are proteins residing and functioning in the cell. Thus, the protein biomarkers of the current invention are normally non-secretory proteins. One would expect that an assay capable of detecting biomarkers in fluid biological samples, such as blood, serum or plasma would be limited to the detection of secretory proteins. Therefore, it is surprising and unexpected that an aspect of the invention is a diagnostic assay and kits for detecting measurable levels of the biomarkers of the invention in fluid biological samples, such as blood, plasma or serum.

In all aspects and embodiments of the present invention, the biomarkers of the invention can be presented as a panel, where a panel refers to the particular biomarker or group of biomarkers that are selected or provided in an assay or method of the invention; or the particular biomarker or group of biomarkers that are provided in a kit of the invention.

In all aspects and embodiments of the present invention, the biomarkers are provided as macromolecules. The macromolecules can be proteins, nucleic acids, antibodies or fragments thereof. When the macromolecule is a protein, fragments can include, but are not limited to, antigenic fragments, N-terminal domains of various lengths, C-terminal domains of various lengths, named domains as identified in the art (such as the Q domain or WD40 domain of Groucho-related proteins). When the macromolecule is a nucleic acid, the nucleic acid can include, but are not limited to, mRNA. cDNA, genomic DNA and fragments thereof. Fragments of nucleic acids can include, but are not limited to, coding sequences and sequences corresponding to the various protein fragments listed above. When the macromolecule is an antibody, the antibodies can include, but are not limited to, polyclonal antibodies, monoclonal antibodies, recombinant antibodies, humanized antibodies and fragments thereof. Fragments of antibodies can include any antigen binding fragment such as, but not limited to, F(ab) fragments and F(ab′)₂ fragments.

As set forth in more detail below, the assay and method according to the present invention could identify patients known to have Stage I or Stage II lung cancer. Identification of patients with early stage lung cancer is particularly valuable as current assays and screening modalities have little ability to do so in a robust and cost effective fashion. The assay is also versatile, by using an assay format that enables testing a large number of samples simultaneously, such as using a microarray, control samples relative to any population can be run in parallel to obtain discriminating data of high confidence, wherein the plurality of controls are matched for as many parameters as possible to the test population.

The present invention also provides a method for determining the treatment therapy that may be effective, based on the biomarker that is changed. Treatment can be selected that is targeted to the biomarker or other proteins that interact with the biomarker.

The present invention also provides for treatment of other cancers such as but not limited to colon cancer in which APC pathway proteins are changed. Treatment may be effected by introducing Groucho-related proteins or homologs or fragments of Groucho-related proteins into a cancer patient in a therapeutically effective amount.

### Biomarker Selection

The selection and identification of lung cancer associated markers, such as protein biomarkers or autoantibodies to the proteins, were done using the Grg1 transgenic mice. Tissue samples from the Grg1 mice were collected and analyzed by methods including, differential RNA arrays, Western blot or proteomic methods such as LC-MS/MS. Levels of protein or gene expression in Grg1 mice were compared to non-tumor bearing control mice. Proteins or genes that were expressed at a different level in the Grg1 mice or in the tumors compared to adjacent normal lung tissue, represent potential lung cancer biomarkers for use in early detection of lung cancer. These proteins were tested for their presence in human lung cancer patient samples.

Antibodies to the biomarkers were compiled on a “diagnostic chip”, e.g., a panel of antibodies was presented on a solid substrate as a microarray and further evaluated for independent predictive value in discriminating samples of lung cancer patients from samples of a non-lung cancer population. Diagnostic markers were selected for the ability to identify the presence of or future presence of radiologically detectable lung cancer in a subject.

### Biomarker Measurement in Patient Samples

As discussed in greater detail below, the invention contemplates the use of different assay formats. Microarrays enable simultaneous testing of multiple markers and samples. Thus, a number of controls, positive and negative, can be included in the microarray. The assay then can be run with simultaneous treatment of plural samples, such as a sample from one or more known affected patients (positive control), and one or more samples from patients without cancer (negative control), along with one or more samples to be tested and compared such as the patient sample. Including internal positive and negative controls in the assay allows for normalization, calibration and standardization of signal strength within the assay. For example, each of the positive controls, negative controls and patient samples can be run in plural, and the plural samples can be a serial dilution. The control sites and patient sample sites also can be randomly arranged on the microarray device to minimize variation due to sample site location on the testing device.

Thus, such a microarray or chip with internal controls enables diagnosis of patients tested simultaneously on the microarray or chip. Such a multiplex method of testing and data acquisition in a controlled manner enables the diagnosis of patients within an assay device as the suitable controls are accounted for and if the panel of markers are those which individually have a reasonably high predictive power, then a point of care diagnostic result can be obtained.

### Exemplification of Sampling and Testing

Samples amenable to testing, particularly in screening assays, generally, are those easily obtainable from a patient, and preferably, in a non-intrusive or minimally invasive manner. A blood sample, plasma or serum is such a suitable sample, and is readily amenable to most immunoassay formats. In the context of a blood sample, there are many known blood collection tubes, many collect 5 or 10 ml of fluid. Similar to most commonly ordered diagnostic blood tests, 5 ml of blood can be collected, but the assay operating as a microarray can require less than 1 ml of blood. The blood collection vessel can contain an anticoagulant, such as heparin, citrate or EDTA. The cellular elements are separated, generally by centrifugation, for example, at 1000*g (RCF) for 10 minutes at 4 C (yielding ˜40% plasma for analysis) and can be stored, generally at refrigerator temperature or at 4 C until use. Plasma samples preferably are assayed within 3 days of collection or stored frozen, for example at −20 C. Excess sample is stored at −20 C (in a frost-free refrigerator to avoid freeze thawing of the sample) for up to two weeks for repeated analysis as needed. Storage for periods longer than two weeks should be at −80 C. Serum samples may be obtained by not providing an anti-coagulant and allowing the sample to clot. Standard handling and storage methods to preserve protein structure and function as known in the art are practiced.

According to the invention, the fluid samples from a human patient suspected of having cancer or in need of ruling out cancer as a diagnosis are then applied to a testing composition, such as a microarray that contain sites loaded with, for example, antibodies for the biomarkers discussed herein, and, in one embodiment, preferably along with suitable positive control and/or negative control samples. The patient, positive control and negative control samples can be provided in graded amounts, such as a serial dilution, to enable quantification. The samples can be randomly sited on the microarray to address any positional effects of the microarray. Following incubation, the microarray is washed and then exposed to a detector. The microarray again is washed, and then in one embodiment, exposed to a reagent to enable detection of a reporter. Thus, if the reporter comprises colored particles, such as metal sols, no particular detection means is needed. In another embodiment of the invention, fluorescent molecules are used and detected with the appropriate incident light. Alternatively, in another embodiment of the invention, enzymes are used and the microarray is exposed to suitable substrates for visualization. The microarray is assessed for reaction product bound to the sites. While that can be a visual assessment, other devices will detect and, if needed, quantify strength of signal. The data then is interpreted to provide information on the validity of the reaction, for example, by observing the positive and negative control samples, and, if valid, the patient samples are assessed. The data obtained from the assay is analyzed to determine whether one or more biomarkers in the patient sample is quantitatively different than in the negative control sample. If the patient sample has a greater or lesser amount of a biomarker, depending on the biomarker(s) chosen, than the negative control sample, the patient is diagnosed as positive for lung cancer.

### Use of the Kit and Assay

The blood assay (test) according to the present invention has multiple uses and applications, although early diagnosis or early warning for subsequent follow up is highly compelling for its potential impact on disease outcomes. The invention may be employed as a diagnostic tool to complement radiographic screening for lung cancer. Serial CT screening is generally sensitive for lung cancer, but tends to be quite expensive and nonspecific (64% reported specificity.) Thus, CT results in a high number of false positives, nearly four in ten. The routine identification of indeterminate pulmonary nodules during radiographic imaging frequently leads to expensive workup and potentially harmful intervention, including invasive biopsies. Currently, age and smoking history are the only two risk factors that have been used as selection criteria by the large screening studies for lung cancer.

The method according to the present invention for detecting radiographically apparent cancers (>0.5 cm) and/or occult or pre-malignant cancer (below the limit of conventional radiographic detection) defines individuals for whom additional screening is most warranted. Thus, the assay according to the invention described herein can serve as a primary screening test, wherein a positive result indicating the presence of cancer is indication for further examination, as is conventional and known in the art, such as further examination by radiographic analysis, such as a CT, PET, X-ray and the like. In addition, periodic retesting may identify emerging non-small cell lung cancer NSCLC.

An example of the clinical application of the assay of the invention described herein is its use in a medical practice where body fluid samples, such as blood, from high risk smokers (for example, persons who smoked the equivalent of one pack per day for twenty or more years) may be assayed for one or more of the lung cancer biomarkers described herein as part of a yearly physical examination. A negative assay result without any further overt symptoms could indicate further testing at least yearly. If the test result is positive, the patient would receive further testing, such as a repeat of the assay and/or a CT scan or X-ray to identify possible tumors by diagnostic imaging. If no tumor is apparent on the CT scan or X-ray, the assay would be repeated once or twice within the year, and multiple times in succeeding years until the tumor is at least 0.5 mm in diameter and can be detected and surgically removed. In addition to its use in a clinical screening protocol, the assay and method of the present invention can also be useful in distinguishing benign nodules from malignant nodules identified on CT screening. A solitary pulmonary nodule (SPN) is defined as a single spherical lesion less than 3 cm in diameter by imaging that is completely surrounded by normal lung tissue. Although the reported prevalence of malignancy in SPNs has ranged from about 10% to about 70%, most recent studies using the modern definition of SPN reveal the prevalence of malignancy to be about 40% to about 60%. The majority of benign lesions are the result of granulomas while the majority of the malignant lesions are primary lung cancer. The initial diagnostic evaluation of an SPN is based on the assessment of risk factors for malignancy such as age, smoking history, prior history of malignancy and chest radiographic characteristics of the nodule such as size, calcification, border (spiculated, or smooth) and growth pattern based on the evaluation of old chest x-rays. These factors are then used to determine the likelihood of malignancy and to guide further patient management.

After an initial evaluation, many nodules will be classified as having an intermediate probability of malignancy (25-75%). Patients in this group would benefit from additional testing with the assay before proceeding to biopsy or surgery. Serial scanning assessing growth or metabolic imaging (e.g. PET scanning) are the only noninvasive options currently available and are far from ideal. Serial radiographic analysis relies on measures of growth, requiring a lesion show no growth over a two-year timeframe; an ideal interval between scans has not been determined although CT scans every 3 months for two years is a conventional longitudinal evaluation. PET scan has 90-95% specificity for lung cancer and 80-85% sensitivity. These predictive values may vary based on regional prevalence of benign granulomatous disease (e.g. histoplasmosis).

PET scans currently cost between $2000 and $4000 per test. Diagnostic yields from non-surgical procedures such as bronchoscopy or transthoracic needle biopsy (TTNB) range from 40% to 95%. Subsequent management in the setting of a nondiagnostic procedure can be problematic. Surgical intervention is often pursued as the most viable option with or without other diagnostic workup. The choice will depend on whether the pretest risk of malignancy is high or low, the availability of testing at a particular institution, the nodule's characteristics (e.g., size and location), the patient's surgical risk, and the patient's preference. Previous history of other extrathoracic malignancy immediately suggests the possibility of metastatic cancer to the lung, and the relevance of noninvasive testing becomes negligible. In the confounding clinical scenario of SPN with indeterminate clinical suspicion for lung cancer, circulating tumor markers could help avoid potentially harmful invasive diagnostic workups and conversely support the rationale for aggressive surgical intervention.

The described invention thus enhances the clinical comfort of electing to serially image a nodule in lieu of invasive diagnostics. The invention also will have an influence in the interval for serial X-ray or CT screening, thereby lowering clinical health care costs. The described invention will complement or supplant PET scanning as a cost effective method to further increase the probability that lung cancer is present or absent.

The invention will be useful in assessing disease recurrence following therapeutic intervention. Blood tests for colon and prostate cancer are commonly employed in this capacity, where marker levels are followed as an indicator of treatment success or failure and where rising marker levels indicate the need for further diagnostic evaluation for recurrence that leads to therapeutic intervention.

Hence, the assay according to the invention described herein is a valuable diagnostic tool for screening, choice of treatment and for continued use during treatment to monitor the course of treatment, success of treatment, relapse, cure and so on. The reagents of the assay, the particular panel of markers can be manipulated to suit the particular purpose. For example, in a screening assay, a larger panel of markers or a panel of very prevalent markers may be used to maximize predictive power for a greater number of individuals. However, in the context of an individual, undergoing treatment, for example, the particular biomarker fingerprint of the patient's specific tumor type can be obtained, which may or may not require all and possibly only a subset of the biomarkers used for screening. The particularized subset of biomarkers can be used to monitor the presence of the tumor in that patient, and subsequent therapeutic intervention.

The components of the assay and kits of the invention can be configured in a number of different formats for distribution and use. For example, the one or more antibodies can be aliquoted and stored in one or more vessels, such as glass vials, centrifuge tubes and the like. The antibody solution can contain suitable buffers and the like, including preservatives, antimicrobial agents, stabilizers and the like, as known in the art. The antibody can be in preserved form, such as desiccated, freeze-dried and so on. The antibodies can be placed on a suitable solid phase for use in a particular assay. Thus, the antibodies can be placed, and dried, in the wells of a culture plate, spotted on a membrane in a layered array or lateral flow immunoassay device, spotted onto a slide or other support for a microarray, and so on. The items can be packaged as known in the art to ensure maximal shelf life, such as with a plastic film wrap or an opaque wrap, and boxed. The assay container can contain as well, positive and negative control samples, each in a vessel, which includes, when a sample is a liquid, a vessel with a dropper or which has a cap that enables the dispensing of drops, sample collection devices, other liquid transfer devices, detector reagents, developing reagents, such as silver staining reagents and enzyme substrate, acid/base solution, water and so on. Suitable instructions for use may be included.

In other formats, such as using a bead-based assay or kit, plural antibodies can be affixed to different populations of beads, which then can be combined into a single reagent, ready to be exposed to a patient sample.

The invention now will be exemplified in the following non-limiting examples.

### EXAMPLES

### Example 1

### Antibody Microarray

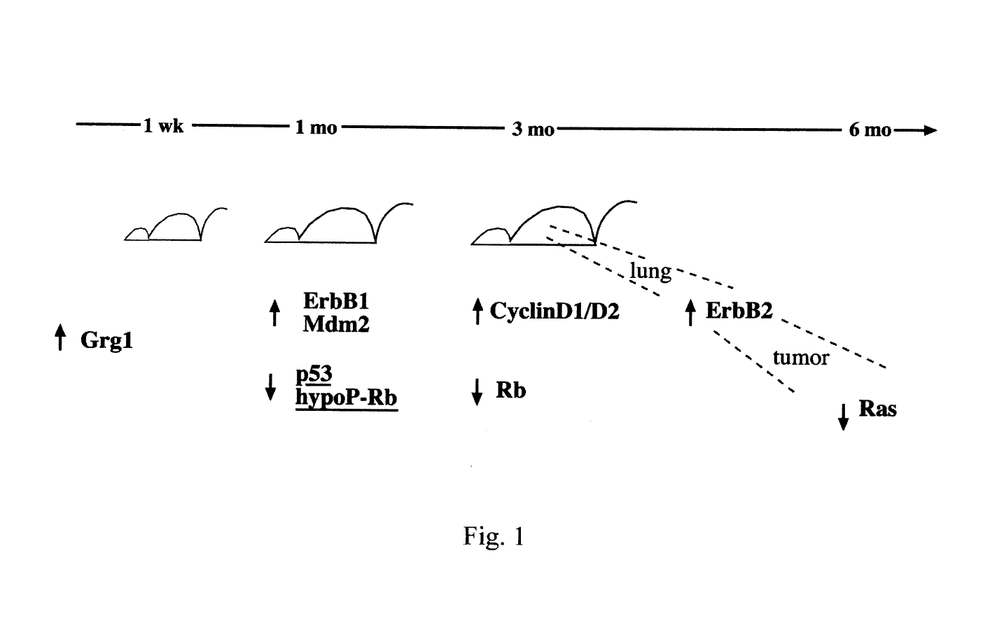

Candidate biomarkers for non-small cell lung cancer were identified in the Grg1 transgenic mouse, using Western blots to measure protein levels in Grg1-expressing mice that develop lung tumors, compared to non-tumor bearing mice. Proteins identified as being changed in the Grg1 transgenic mice are shown in FIG. 1.

The aim of the antibody microarray experiment is to evaluate the relative abundance of 10 human proteins in lung cancer versus control samples, using a custom antibody array for 30 human serums.

### Spotting Protocol and Incubation of the Slides

### 1. Spotting of the Slides

The spotting was done by the Microgrid II spotter with Quill Pins Microspot 2500. The spot diameter is 150 μm. The spotter is equipped by a temperature controlled Biobank set at 4° C.

The slides used were nitrocellulose slides (Gentel PATH PLUS Protein Microarray slides).

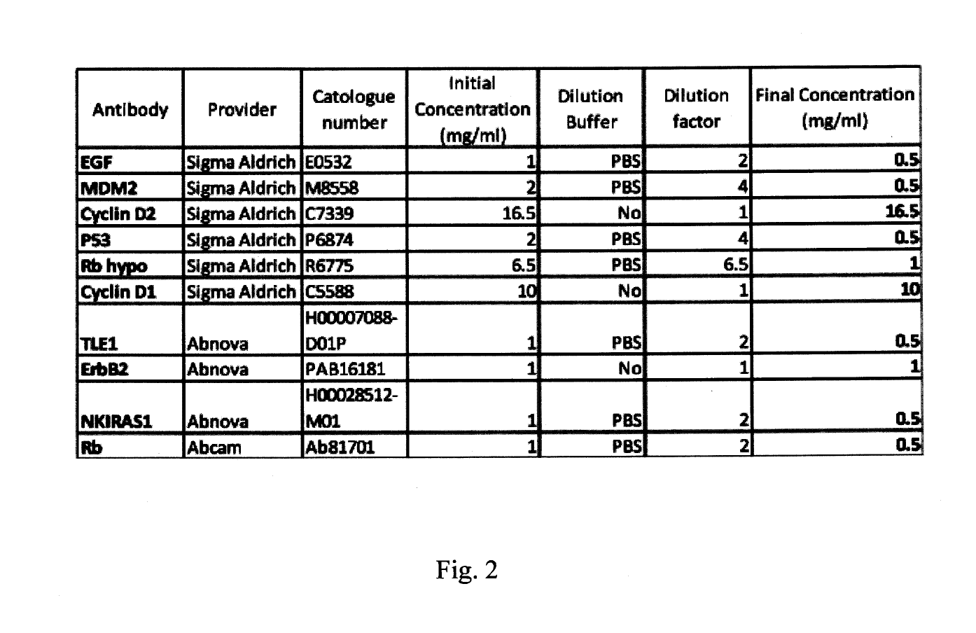

The antibodies were spotted on 14 pads in their commercial storing buffer or diluted with Phosphate Buffer Saline (0.01M, pH7.4). The dilutions used for each antibody were optimized (see FIG. 2). Three replicates of each antibody were spotted per array.

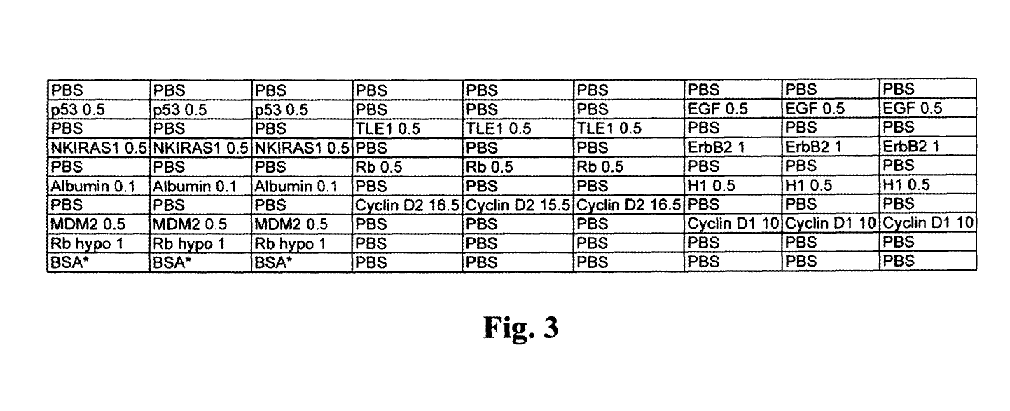

There were 4 different controls spotted in triplicate in each pad (see FIG. 3). The first, PBS (Phosphate Buffered Saline), is a negative control. The second, labeled BSA (spotted in 0.05 mg/ml) is a grid positioning/positive control. The third is an anti-Albumin antibody (spotted in 0.1 mg/ml) which is a positive control of serum incubation. The last control is an Anti-H1 antibody (Anti-Histone H1, spotted at 0.5 mg/ml) which is a negative control.

### 2. Labelling and Incubation of Samples

Serum samples were obtained from Asterand: Asterand XPressBANK™ human serum from lung cancer patient donors and human normal control serum. The human normal control serum corresponds to a reference sample as described herein. The stage of each patient's cancer was determined according to tumor histology.

500 μl of each serum (diluted at 1/10) were labelled with 50 μl of Sulfo-NHS-LC-Biotin 40 mM. Mixes were incubated for 1 hour at room temperature with short vortexing every 10 minutes. The protocol has been optimized to obtain the best signal/noise ratio.

Slides were washed 2 times 2 minutes with shaking (50 rpm) and blocked with 5 ml of PBS (0.01M; pH=7.4) Tween 0.1% Casein 1% with gentle shaking (15 rpm) during 1 hour at room temperature.

70 μl of each sample was incubated at 1/300 (PBS+Tween0.05%+Casein 0.1%) during 1 h at room temperature with gentle shaking. Then, each well was washed 5 times 5 minutes with 80 μl of PBS+Tween 0.05% Casein 0.1% at 35 rpm.

70 μl of Streptavidin AlexaFluor 647 solution was incubated at 1 μg/ml in each well during 1 h at room temperature in a dark place with gentle shaking (15 rpm). Each well was washed 5 times 5 minutes with 80 μl of PBS+Tween 0.05% Casein 0.1% at 35 rpm in a dark place and the slides were washed 2 times with 5 ml of PBS+Tween 0.05% Casein 0.1% during 5 minutes at 35 rpm in a dark place. Finally, slides were rinsed with distilled water and are dried in a dark clean place and scanned.

### Image Analysis

Slides were scanned with Innoscan700 fluorescent MicroArray scanner. (Innoscan 700—Innopsys™). Scan parameters were optimized.

Images were processed with the software Mapix© (Vers. 2.8.2). Detection grids and spot identification were overlaid to corresponding images.

### Analysis Parameters

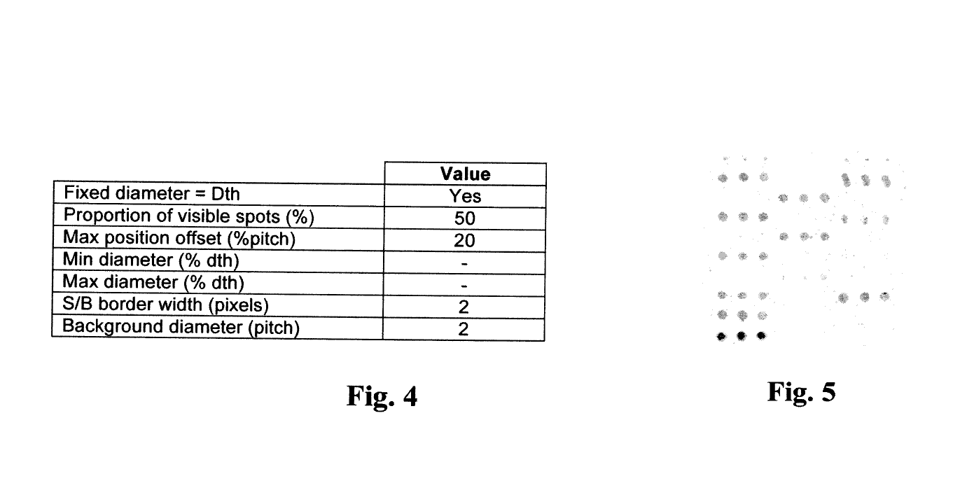

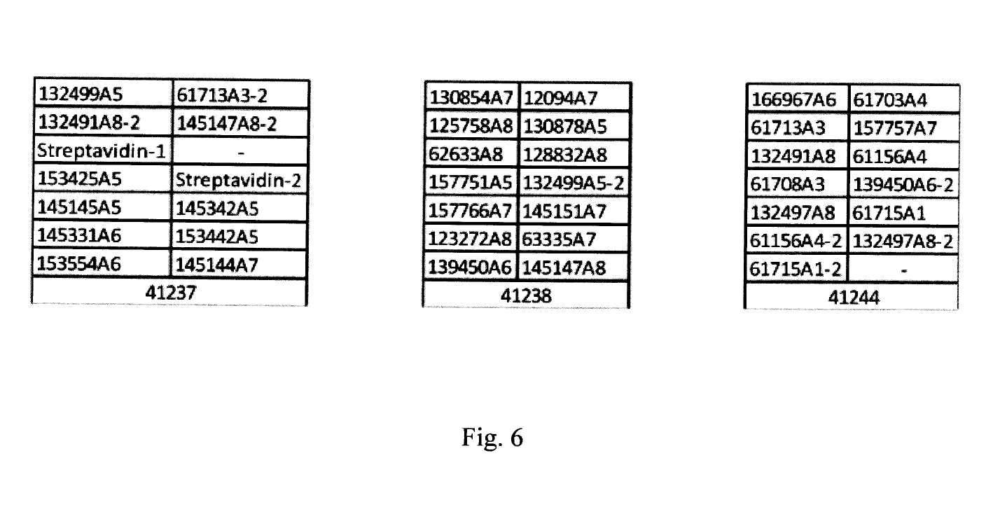

The software dedicated to the image analysis allows setting up the spot detection parameters (spot detection threshold, variable or constant spot diameter, anomaly detection). The parameters set up for this analysis with Mapix are shown in FIG. 4. An example image is shown in FIG. 5. The incubation map is displayed in FIG. 6.

The images were also visualized in order to check the detection grid position and to detect the presence of anomalies or erroneous spot detection (dust detection, high background). Some samples have been incubated twice. The best images have been chosen for each of these samples.

### Quality Control

### 1. Spotting Controls

The first quality control was made visually. It revealed no anomaly that could affect the analysis.

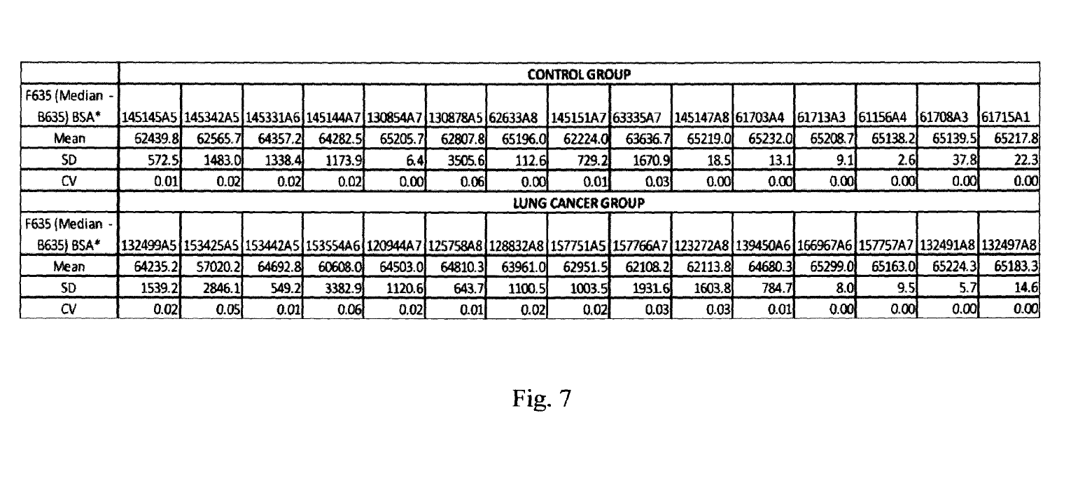

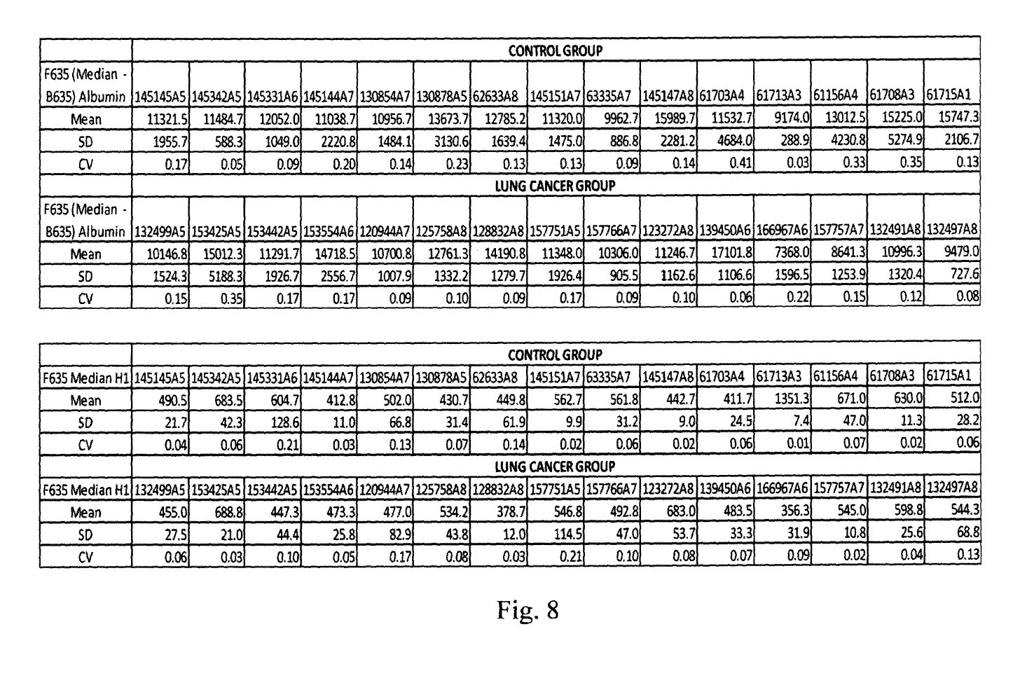

Each grid contained positioning positive control spots: labeled BSA (3 spots per pad) and anti-Albumin antibody (3 spots per pad). Labeled BSA spots, which were the last spotted, were used for the grid positioning and control that all spots were spotted (FIG. 7). Anti-Albumin antibody is a serum incubation control on the microarray. As expected this positive control showed high reactivity (FIG. 8).

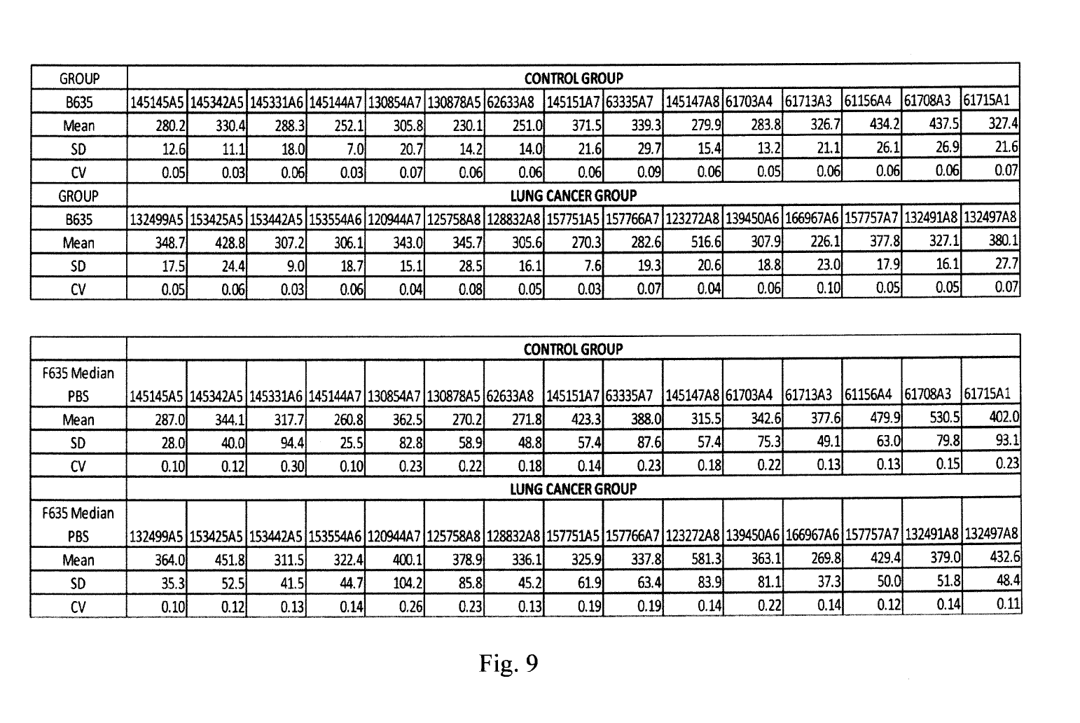

There were also negatives control spots: PBS and anti-H1 antibody. PBS (51 per grid) was used to check for non-specific signal. The corresponding signals were expected to be about the background level (FIG. 9). The anti-H1 antibody is a negative control for the incubation of serum. As expected negative controls showed low reactivity (FIG. 8).

### 2 Incubation Control

Two Arrays were incubated with the incubation buffer instead of a patient sample to check the non-specific signals obtained with only the incubation of Streptavidin Alexafluor solution.

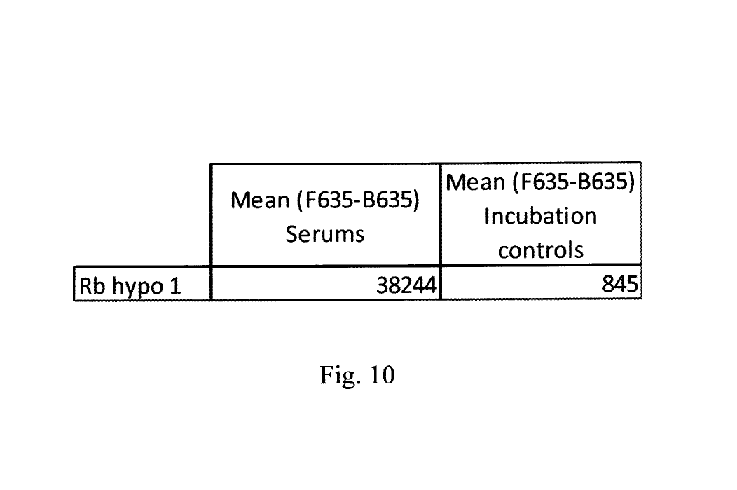

All the signals measured with this control were near the background (under the background proximity cut off) except for the “Rb hypo” antibody. For this antibody, the signal obtained was above the cut-off but was very low in comparison with its signal measured when a sample was incubated (FIG. 10).

### Raw Data Treatment

The intensity of each spot was calculated from the median value of the pixels contained in the spot.

The measured signal for a spot is affected by the local background (measured in the area surrounding the spot) that increases the signal. Thus, the local background is systematically subtracted from the signal to obtain a net signal. The net signal may be negative due to a background level a little higher than the spot signal (weak spot signal). In this case, the negative net values (Median Spot Signal−Median Local Background Signal) are replaced by the value “10”: revised value.

Each antibody was spotted in triplicate on each array. This allows checking the signal validity verifying the values concordance between replicates. If the triplicates present a CV>50%, then the calculated median value is highlighted with an asterisk (*) in the table and in the corresponding normalized values.

The spots presenting a signal level near the background have an important sensitivity to the background variation. So, comparison of a couple of data near the background may result in great ratios despite low signals. In order to clearly identify these values, we set a background proximity cut-off for each spot calculated as follows:

Mean(of pixels)of Local Background+3SD

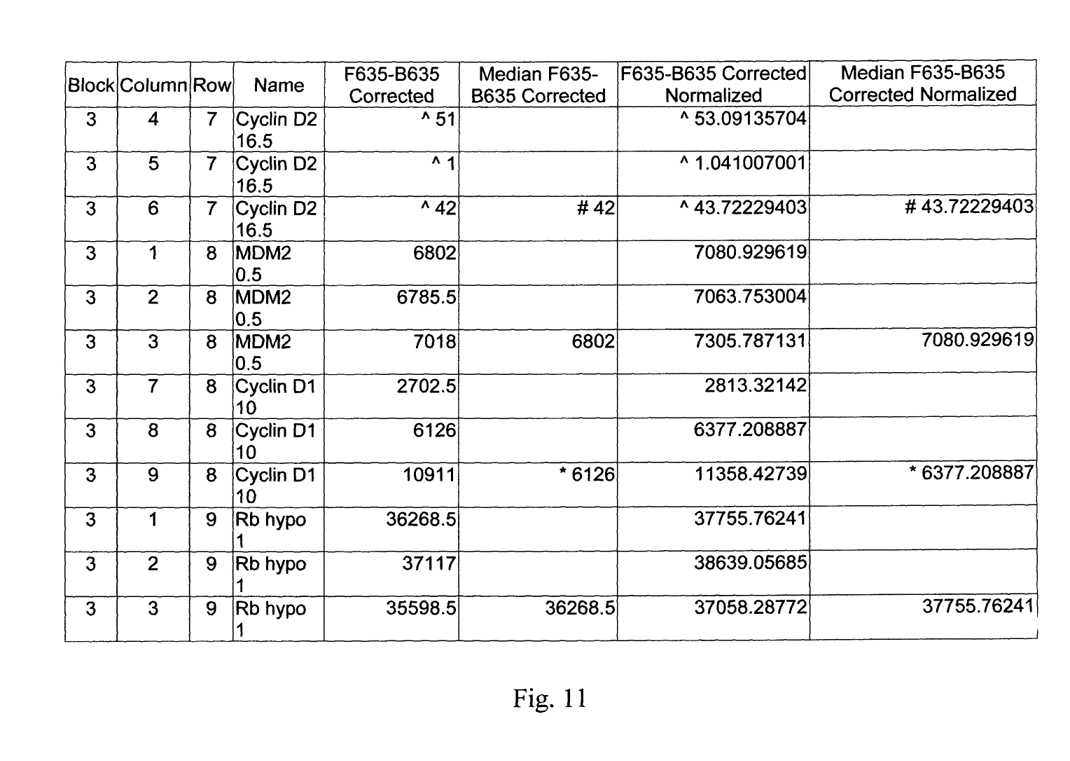

When one replicate presented a signal under the cut-off, its net signal value is highlighted with a carat (̂) in the table. If at least two on the three replicate of a spot present a signal under the cut-off, the protein signal is considered as near the background. In this case, the median value calculated from the triplicates is highlighted with a hatch sign (#) in the table, independently of the value of the CV between the spots triplicate. The highlight is reported on the corresponding normalized values (example, FIG. 11).

### Analysis and Normalization

To compare the populations of signals obtained with different samples and slides, a normalization of each population is necessary. Indeed, experimental parameters may vary and protein concentrations are not exactly the same.

The normalization on the mean of intensities has then been chosen. The reference for the normalization was calculated by calculating mean of intensities of all spots (net signals) for each sample except for control spots.

The signal for each protein was then normalized as follows:

Normalized Value(VN)=(Signal−Bkg)antibody/Mean(signals−Bkg))array×K

K: constant (for data reading comfort)=10 000

The different analyses described below are performed with these normalized values.

### Comparison Control Group Vs Lung Cancer Group

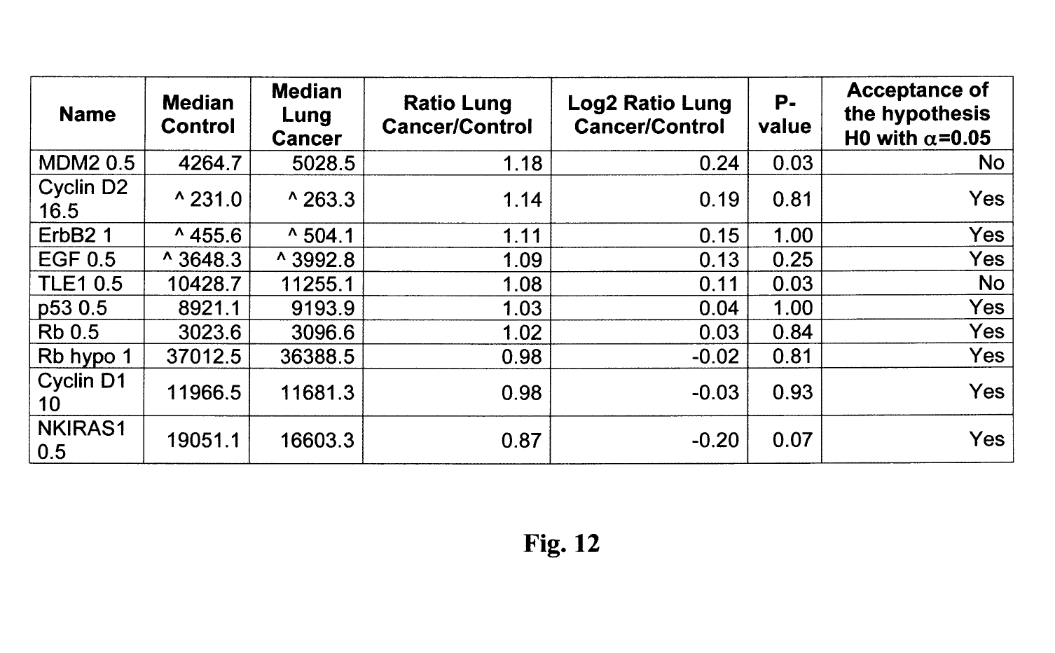

There were 2 groups of samples: CONTROL and LUNG CANCER. The following comparison was made: LUNG CANCER/CONTROL (FIG. 12). Each group contained 15 samples and the medians of each group were calculated. The ratios (median LUNG CANCER group)/(median CONTROL group) and Log 2 (Ratio) were calculated from the normalized values.

The resulting median value of a group is also highlighted with a carat (̂) in FIG. 12 if at least half of the values in a group were highlighted.

In order to compare the groups of samples, a Mann & Whitney test was also used Mann & Whitney is a non-parametric test used for the independent samples. The null hypothesis for the test is H0: the population medians are equal. The level of significance chosen for the test was 5% (α<0.05).

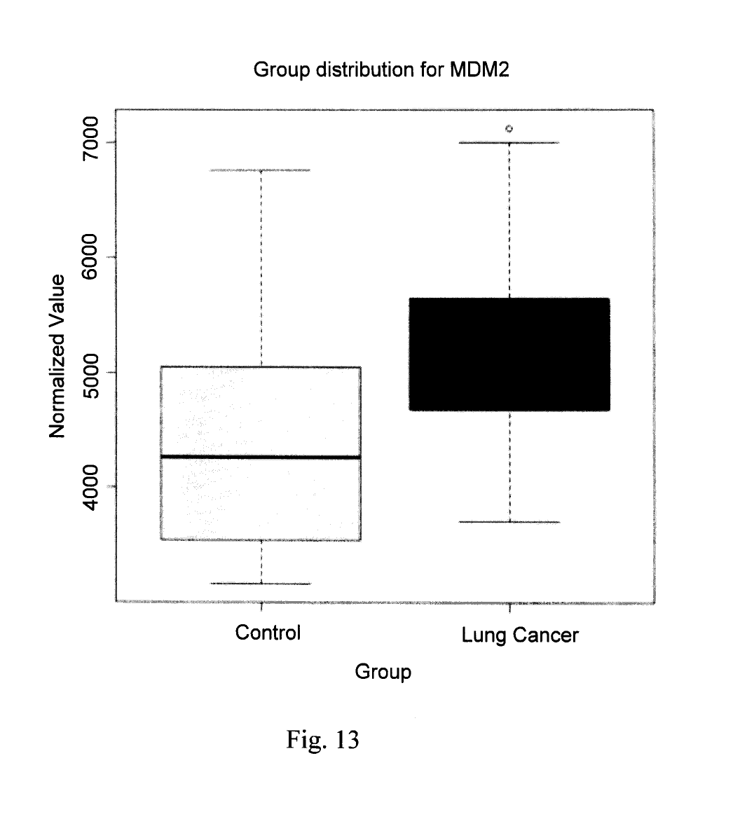

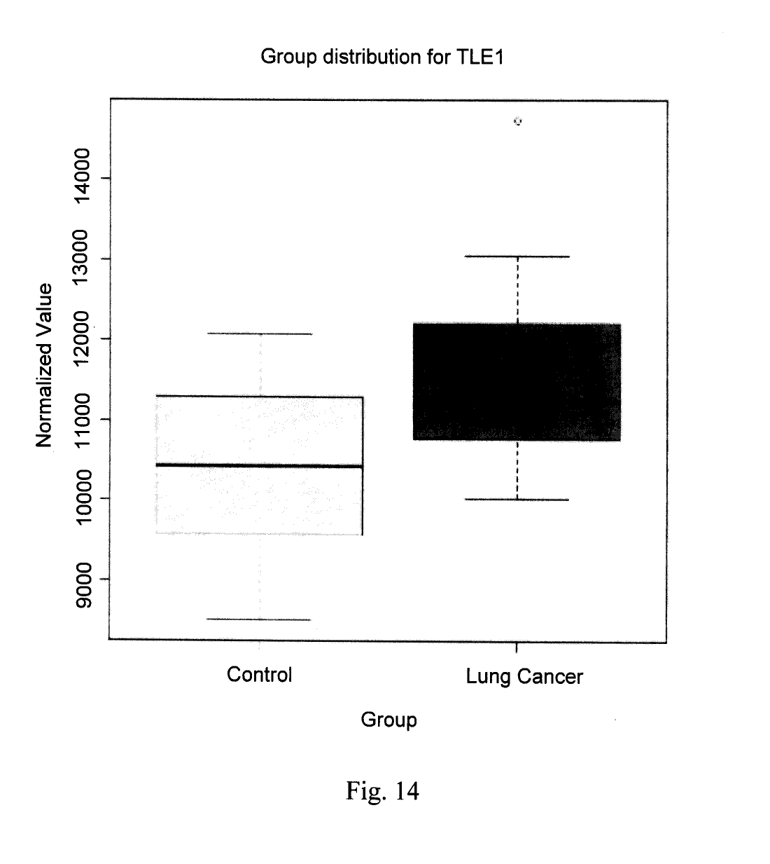

The values of ratio obtained for all the antibodies were relatively weak but the statistical test of Mann & Whitney permitted isolation of 2 antibodies: MDM2 (FIG. 13) and TLE1 (FIG. 14) (Hypothesis H0 is rejected). For these 2 antibodies, proteins are over expressed in the Lung Cancer Group in comparison to the Control Group.

### Conclusion

The data analysis revealed 2 potentially differentially expressed proteins using the Mann & Whitney test: MDM2 and TLE 1. These 2 proteins are over expressed in the Lung Cancer group compared to the Control group.

### Example 2

### Drug Treatments

The biomarkers can be used to predict the effectiveness of a drug treatment. The Grg1 transgenic mice overexpress the Grg1 (human TLE1) protein, which interacts with HDAC complexes to carry out its function. Thus, the lung tumors in the Grg1 transgenic mice might be inhibited by treatment with an HDAC inhibitor. HDAC inhibitor drugs are used in the treatment of many cancers, but it is not clear which patients will respond. Furthermore, HDAC inhibitors have severe side effects, therefore HDAC inhibitors with more specific activity are being developed. The Grg1 mouse model provides an ideal setting to test new HDAC inhibitors for effectiveness against non-small cell lung cancer.

Trichostatin A (TSA) is a histone deacetylase inhibitor that inhibits the proliferation of lung carcinoma cell lines. The effect of TSA on lung cancer development in animal models, however, has not been investigated. In the present example, TSA was used to treat transgenic mice that develop branchioalveolar lung carcinomas due to overexpression of Grg1. Upon TSA treatment, it was discovered that lung tumor growth was inhibited in Grg1 transgenic mice. In the group of TSA treated Grg1 transgenic mice, only 1 out of 6 developed tumors. In addition, tumor angiogenesis was also inhibited by TSA. These findings indicate that TSA can effectively inhibit lung tumor growth in viva and inhibition of histone deacetylase activity has therapeutic potential towards branchioalveolar adenocarcinoma.

### Materials and Methods

Transgenic mice were generated with Cre-conditional expression of Grg1 (murine TLE1). The transgene initially expresses a lacZ reporter gene. When Cre recombinase is introduced, the loxP-flanked lacZ gene is excised, and the transgene expresses Grg1 and another reporter gene, human placental alkaline phosphatase (hPLAP). Widespread expression of the transgene both before and following Cre excision was demonstrated and it was found that Grg1 overexpression resulted in development of lung adenocarcinomas.

The mice expressing the lacZ reporter prior to Cre excision are referred to as Grg1^{lacZ} mice, and the mice expressing Grg1 and hPLAP following Cre excision are referred to as Grg1^{hPLAP} mice. This disclosure relates only to the Grg1-expressing (Grg1^{hPLAP}) mice, and therefore they are referred to alternatively as Grg1hPLAP mice or simply as Grg1 transgenic mice.

Grg1 transgenic mice on 129 background were generated and were genotyped by alkaline phosphotase staining as described, Allen, T. et al. “Grg1 Acts as a Lung-Specific Oncogene in a Transgenic Mouse Model”, Cancer Res, 66:3, 1294-1301 (2006). TSA (BIOMOL Research Laboratories, Plymouth Meeting, Pa.) was injected intraperitoneally into 1-month old Grg1hPLAP mice and control littermates at 0.5 mg/kg in 40 μl of 10% DMSO/PBS (Mishra et al., 2003). A control solution of 40 μl of 10% DMSO/PBS was injected to other groups of Grg1 mice and control littermates. Each group consisted of 6 male mice and the injections continued for 30 days. After 3 months, the mice were sacrificed and the lungs were dissected for histological examination. The inferior lobes of the left lung were fixed in 4% paraformaldehyde and later embedded in paraffin for serial sectioning of 5 μm at 100 μm step intervals throughout the lobe. The sections were stained with H&E and were screened for adenomatous/carcinomatous proliferation at 10× magnification. The number of the lesions was recorded and statistical significance was measured by ANOVA.

### Results

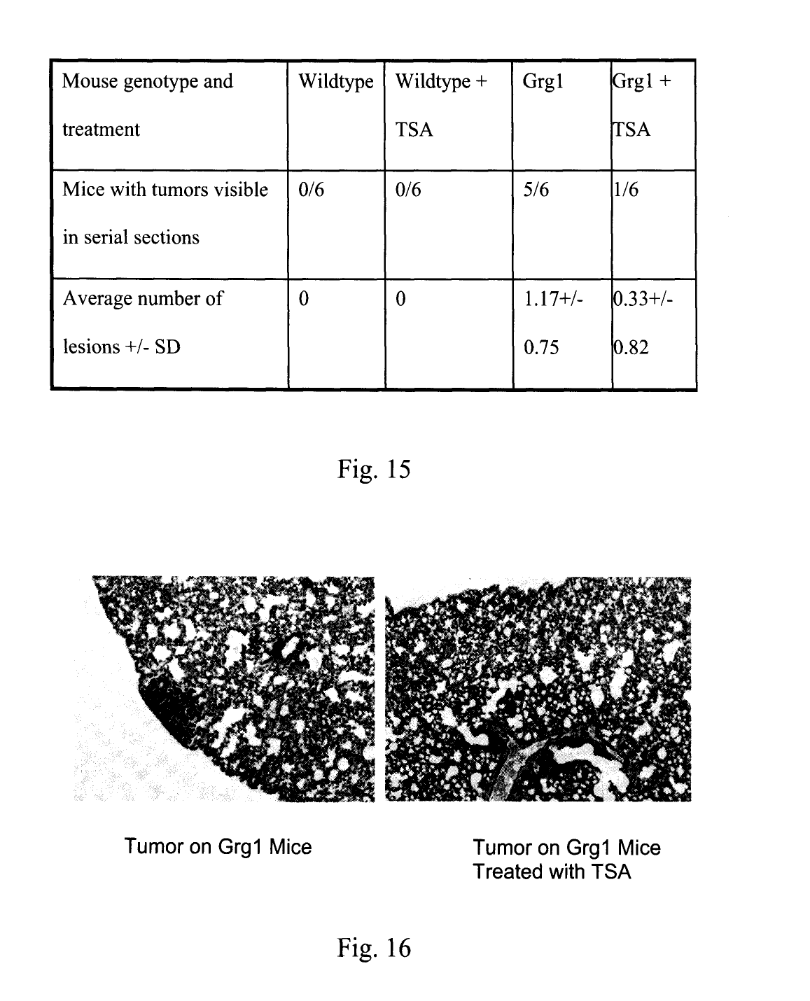

At 5 months of age the Grg1 overexpressing mice developed tumors visible on serial sections. The group of TSA-treated Grg1 transgenic mice were healthy and the histological sections showed a significant decrease in tumor growth (FIG. 15). Only 1 of the 6 TSA treated Grg1 mice developed a tumor, compared with the untreated group in which 5 out of 6 Grg1 mice developed tumors. Non-transgenic animals did not develop tumors or show any obvious phenotype with or without TSA treatment.

Examination of H&E stained serial sections revealed that the tumors which developed in the Grg1 overexpressing mice showed extensive proliferation and blood vessels were observed to grow into the tumors. FIG. 16 shows the lesions from the single mouse which developed tumors following TSA treatment. The lesion has similar cellular morphology but was a much smaller size. No blood vessels were observed around the lesion. No other abnormalities were observed on the lung tissue from TSA treated Grg1 transgenic mice and non-transgenic mice.

### Example 3

### Groucho Proteins can be Used to Treat Tumors

Considering the proposed antagonistic role for Grg proteins with respect to beta-catenin/Tcf function, Grg proteins might serve a tumor suppressor role with respect to malignancies caused by aberrant Wnt/beta-catenin signaling. However the lung tumor phenotype of Grg1 is contradictory to this hypothesis. To address this issue further, Grg1 and Grg5 overexpressing mice were crossed to mice carrying the APC^{min} allele. The APC^{min/+} mice carry a truncating mutation in codon 850 of one copy of the murine APC gene. Loss of the wildtype APC allele results in elevated levels of beta-catenin and the formation of intestinal adenomas in these mice. We found that Grg1 overexpression has a significant effect on the development of intestinal polyps in the APC^{min/+} model. Conversely, the presence of the APC^{min} allele also has consequences for lung adenomas induced by Grg1.

### Grg1 Decreases the Number of Macroadenomas in APC^{min/-} Mice

On a C57BL/6J background APC^{min/+} mice have a life span of 4 to 6 months and develop multiple intestinal adenomas. Adenoma multiplicity and lethality are reduced on an outbred background due to the presence of modifier loci. We crossed C57BL/6J APC^{min/+} mice to Grg1 and Grg5 mice to produce mice that carry the APC^{min} allele and also overexpress Grg1 or Grg5. In order to rule out effects associated with transgene insertion, APC^{min/+} mice were also crossed to Grg1^{lacZ} and Grg5^{lacZ} mice to produce mice with the APC^{min} allele plus non-excised transgenes. The outbred background of mice produced by these crosses allowed for the assessment of mice at a time point of 6 months and simultaneous examination of Grg1-associated lung tumors and APC^{min/+}-associated intestinal polyps. We found that this time point was ideal for the quantitation of lung adenomas in Grg1 overexpressing mice.

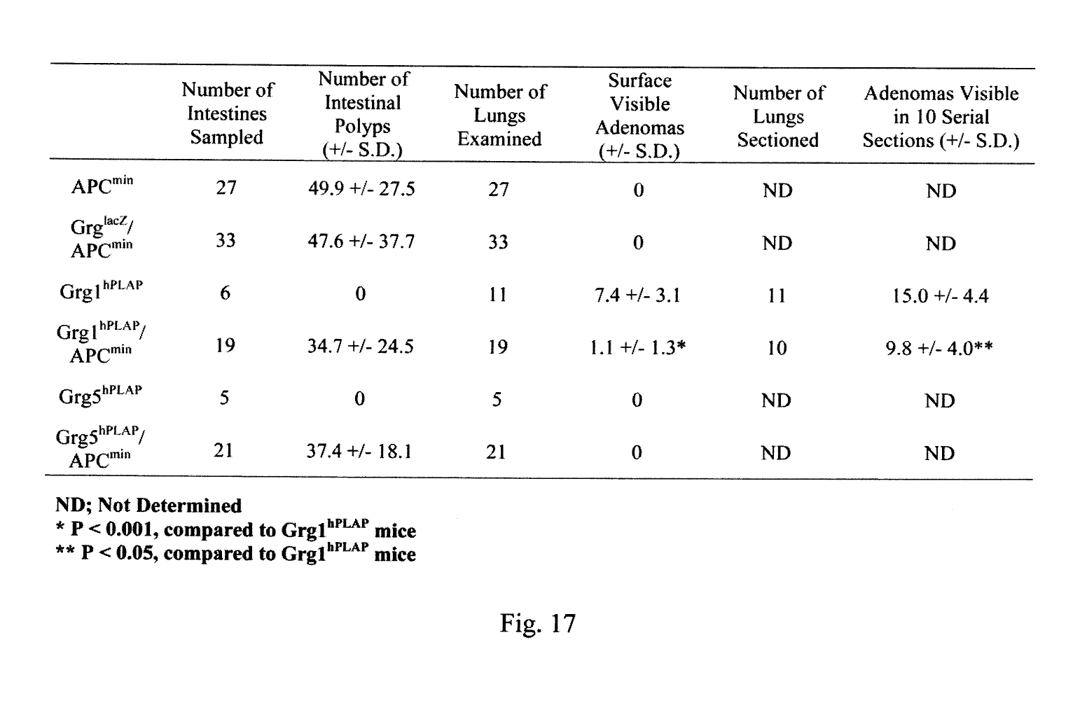

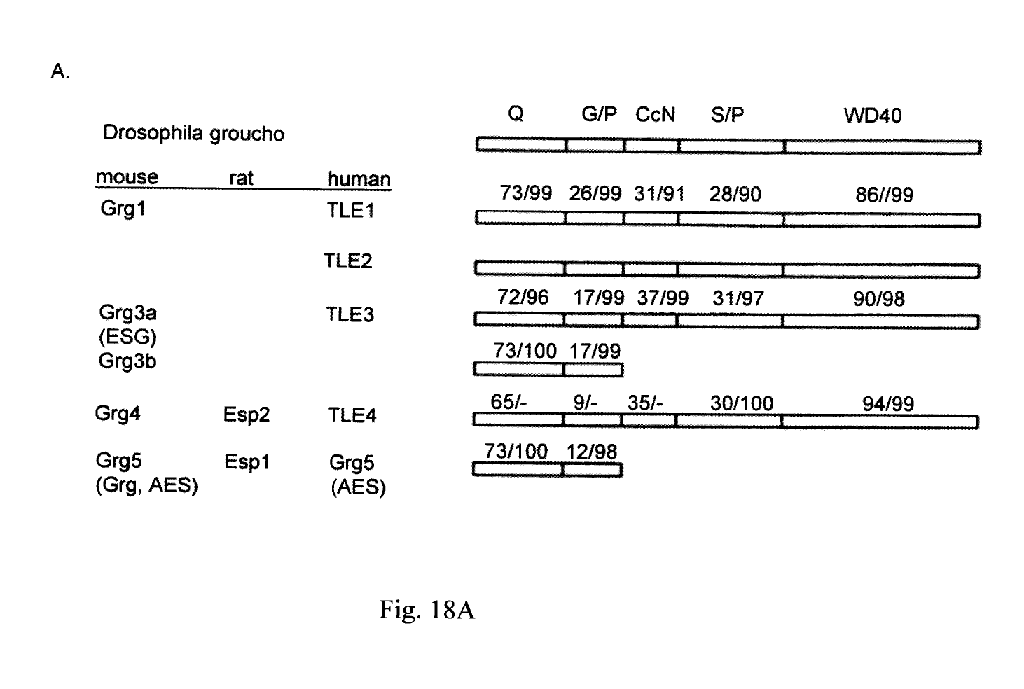

The small intestine and colon of mice were removed in entirety and the number and size of intestinal polyps was measured. FIG. 17 shows the average number of intestinal polyps in mice with each of the genotypes examined. No polyps were found in Grg1 (n=6) or Grg5 (n=5) mice that do not carry the APC^{min} allele. In APC^{min/+} mice an average of 49.9 polyps developed and similarly in control Grg^{lacZ}/APC^{min/+} mice there were an average of 47.6 polyps. The total number of polyps was not significantly altered in Grg1/APC^{min/+} or Grg5/APC^{min/+} mice with Grg1 or Grg5 overexpression, although there was a trend towards a lower number of polyps than in APC^{min} mice.

Lethality in C57BL/6J APC^{min/+} mice is usually associated with intestinal obstruction due to the progression of one or more adenomas. The size of adenomas is therefore an important determinant of disease severity. We measured the size of intestinal polyps that developed in APC^{min/+} mice in the absence or presence of Grg1 and Grg5 overexpression. The number of macroadenomas (>2 mm) found in APC^{min/+} mice that overexpress Grg1 was significantly reduced. Grg5/APC^{min/+} mice did not show a similarly robust decrease in the number of large intestinal adenomas. Therefore overexpression of Grg1, a full length Groucho protein, reduced the number of macroadenomas. The body mass, length of the small intestine and length of the colon of Grg1/APC^{min/+} mice were not significantly different compared to the other groups examined.

### Lung Tumor Burden is Lowered in Grg1 Mice Carrying the APC^{min} Allele

Grg1 mice develop 8.3+/−6.0 (n=28) lung lesions visible on the pleural surface at 180 days. Crossing one generation into the C57BL/6J background did not yield significant alterations in lung adenoma number indicating there were no strain specific effects on the Grg1-induced phenotype (FIG. 17, column 5; 7.4+/−3.1 surface visible adenomas). However, the presence of the APC^{min} allele dramatically reduced the number of lesions that could be found on the pleural surface and in lung serial sections (FIG. 17, columns 5, 7). In general, adenomas in Grg1 mice were larger than those found in Grg1/APC^{min/+} mice. Examination of the adenomas at high magnification revealed no visible differences in cell morphology between the two genotypes. Therefore, although the APC^{min} allele does not completely abolish the induction of lung adenomas by Grg1, lung tumor burden was significantly reduced.

### Materials and Methods

### Mouse Lines and Genotyping

APC^{min/+} mice were produced and maintained on a C57BL/6J background from stock obtained from The Jackson Laboratory (Bar Harbor, Me.). The iZ/AP-Grg1 (line 2F12) and iZ/AP-Grg5 (line H2) mice have been described in Allen, T. et al. “Grg1 Acts as a Lung-Specific Oncogene in a Transgenic Mouse Model”, Cancer Res, 66:3, 1294-1301 (2006). Mice with the non-excised transgenes are referred to as Grg1^{lacZ} and Grg5^{lacZ}. Mice with globally excised iZ/AP-Grg transgenes are referred to as Grg1 or Grg5. To create mice carrying the APC^{min} allele and iZ/AP-Grg transgenes, C57BL/6J, APC^{min/+} mice were crossed with F2 and F3 generation iZ/AP-Grg transgenic mice that carry either non-excised (Grg^{lacZ}) or excised (Grg) transgenes. Genotyping for the APC^{min} allele was done by competitive PCR using primers and conditions as described Dietrich, W. F. et al “Genetic identification of Mom-1, a major modifier locus affecting Min-induced intestinal neoplasia in the mouse”, Cell, 75:4, 631-639 (1993). For the presence of Grg1 and Grg5 transgenes, mice were genotyped by either lacZ or hPLAP staining of ear punch tissue.

### Quantitation of Intestinal Polyps and Lung Adenomas

Animals were sacrificed at approximately 6 months of age. The small intestine and colon were removed in entirety from each mouse and flushed of their contents with PBS. Intestines were fixed in 4% paraformaldehyde/PBS overnight. The next day intestines were washed in PBS and stored in 70% ethanol (v/v). For examination, fixed intestines were opened longitudinally and pinned lumen side up to a layer of hardened 3% agarose in a petri dish. With the use of a dissecting microscope (20-40×) and calipers, both polyp number and size were scored for the entire small intestine and colon. Polyp sizes were determined by measuring the maximum diameter of each polyp. Intestinal polyps with a diameter of as little 0.3 mm could be scored in this fashion. Lungs from the same mice were examined for tumors on the pleural surface using a dissecting microscope (10×). Serial sectioning of paraffin-embedded lung was done to inspect for tumors not visible on the pleural surface. 5 μm sections were cut at 100 μm intervals through the left lobe. Sections were stained with hematoxylin and eosin and visually scanned for microadenomas at 100× magnification. Adenomas large enough to be present on adjacent sections were not counted twice. Ten serial sections were scored per mouse.

### Statistical Analysis

An analysis of variance (ANOVA) was carried out to test for significant alterations in polyp multiplicity, small intestinal length, colon length and weight. The non-parametric Kruskal-Wallis test followed by Dunn's test was used to assess for significant differences in polyp size. The statistical significance of alterations in tumor number on the pleural surface or in lung serial sections was analyzed using the Mann-Whitney test.

### Western Blotting

Whole tissue protein extracts were made from freshly dissected tissues using a cold lysis buffer of 150 mM NaCl, 25% glycerol, 0.2 mM EDTA, 20 mM Hepes pH 7.8, 0.5 mM DTT, 0.5 mM PMSF, 0.5 mg/ml Leupeptin, 0.7 mg/ml Pepstatin, 2 mg/ml Aprotonin. Tissues were sonicated and debris was removed by brief centrifugation. Supernatant was transferred to a fresh tube and immediately frozen at −80° C. Protein concentrations were measured using the Bradford method (Bio-Rad). For Western blot analysis, 100 μg of lysate was run on a 10% polyacrylamide gel and transferred to PVDF membrane. Blocking was done overnight in 10% skim milk powder in 10 mM Tris HCl pH8.0, 150 mM NaCl, 0.1% Tween (TBST). Binding of primary and secondary antibodies was in 5% skim milk powder TBST. Rabbit polyclonal serum directed against Grg5 was created using a C-terminal 18 amino acid peptide. Primary commercial antibodies were α-TLE1 rabbit polyclonal serum M-101 (Santa Cruz Biotechnology) (Husain et al., 1996), mouse monoclonal α-beta-galactosidase (Promega), mouse monoclonal α-beta-actin AC-15 (Sigma), α-beta-catenin H-102 rabbit polyclonal serum (Santa Cruz Biotechnology), mouse monoclonal α-active beta-catenin (α-ABC) 8E7 (Upstate), and α-phospho-beta-catenin (Ser-33, 37, Thr-41) rabbit antiserum (Cell Signaling). HRP-conjugated secondary antibodies were purchased from Santa Cruz Biotechnology.

### Immunostaining

Following deparaffination, rehydration and antigen retrieval, sections were incubated with α-Grg3/TLE2 rabbit polyclonal serum (Santa Cruz Biotechnology), α-Grg1/TLE1 rabbit polyclonal serum, α-beta-catenin rabbit polyclonal serum, α-ABC mouse monoclonal antibody or α-phospho-beta-catenin (Ser-33, 37, Thr-41) rabbit antiserum. Sections were subsequently incubated with biotinylated secondary antibodies, Avidin-Biotin Complex and DAB (Vector Laboratories Inc.). Sections were counterstained with hematoxylin. For immunofluorescence, a goat α-mouse tetramethylrhodamine-conjugated secondary antibody (Molecular Probes) was used.

### Histochemical Staining

Fixation, and staining of tissues and frozen sections was essentially as described in Lobe C. et al. “Z/AP, a double reporter for Cre-mediated Recombination”, Dev. Biol. 208:2 281-292 except that no counterstain was used on lacZ or hPLAP stained frozen sections.

SEQUENCE LISTING

Human TLE1 protein
SEQ ID NO: 1
mfpqsrhptp hqaagqpfkf tipesldrik eefqflqaqy
hslklecekl asektemqrh yvmyyemsyg lniemhkqte
iakrintica qvipflsqeh qqqvaqaver akqvtmaeln
aiigqqqlqa qhlshghgpp vpltphpsgl qppgipplgg
sagllalssa lsgqshlaik ddkkhhdaeh hrdrepgtsn
sllvpdslrg tdkrrngpef sndikkrkvd dkdsshydsd
gdksddnlvv dvsnedpssp raspahspre ngidknrllk
kdassspast assasstslk skemslheka stpvlksstp
tprsdmptpg tsatpglrpg lgkppaidpl vnqaaaglrt
plavpgpypa pfgmvphagm ngeltspgaa yaslhnmspq
msaaaaaaav vaygrspmvg fdppphmrvp tippnlagip
ggkpaysfhv tadgqmqpvp fppdaligpg iprharqint
lnhgevvcav tisnptrhvy tggkgcvkvw dishpgnksp
vsqldclnrd nyirsckllp dgctlivgge astlsiwdla
aptprikael tssapacyal aispdskvcf sccsdgniav
wdlhnqtivr qfqghtdgas cidisndgtk lwtggldntv
rswdlregrq lqqhdftsqi fslgycptge wlavgmessn
vevlhvnkpd kyqlhlhesc vlslkfaycg kwfvstgkdn
llnawrtpyg asifqskess svlscdisvd dkyivtgsgd
kkatvyeviy
Human TLE2 protein
SEQ ID NO: 2
mypqgrhptp lqsgqpfkfs ileicdrike efqflqaqyh
slklecekla sektemqrhy vmyyemsygl niemhkqaei
vkrlsgicaq iipfltqehq qqvlqavera kqvtvgelns
ligqq1qpls hhappvpltp rpaglvggsa tgllalsgal
aaqaqlaaav kedragveae gsrverapsr saspsppesl
veeerpsgpg gggkqradek epsgpyesde dksdynlvvd
edqpseppsp attpcgkvpi ciparrdlvd spaslasslg
splprakeli lndlpastpa skscdssppq dastpgpssa
shlcglaakp apstdsvalr spltlsspft tsfslgshst
ingdlsvpss yvslhlspqv sssvvygrsp vmafeshphl
rgssyssslp sipggkpays fhvsadgqmq pvpfpsdalv
gagiprharq lhtlahgevv cavtisgstq hvytggkgcv
kvwdvgqpga ktpvaqldcl nrdnyirsck llpdgrsliv
ggeastlsiw dlaaptprik aeltssapac yalayspdak
vcfsccsdgn ivvwdlqnqt mvrqfqghtd gascidisdy
gtrlwtggld ntvrcwdlre grqlqqhdfs sgifslghcp
nqdwlavgme ssnveilhvr kpekyqlhlh escvlslkfa
scgrwfvstg kdnllnawrt pygasifqsk esssvlscdi
srnnkyivtg sgdkkatvye vvy
Human TLE3 protein
SEQ ID NO: 3
mypqgrhpap hqpgqpgfkf tvaescdrik defqflqaqy
hslkveydkl anektemqrh yvmyyemsyg lniemhkgte
iakrintila qimpflsqeh qqqvaqaver akqvtmteln
aiigqqqlqa qhlshathgp pvqlpphpsg lqppgippvt
gsssgllalg algsqahltv kdeknhheld hreressann
syspseslra sekhrgsady smeakkrkae ekdslsryds
dgdksddlvv dvsnedpatp rvspahsppe ngldkarslk
kdaptspasv asssstpssk tkdlghndks stpglksntp
tprndaptpg tsttpglrsm pgkppgmdpi gimasalrtp
isitssyaap fammshhemn gsltspgaya glhnippqms
aaaaaaaaay grspmvgfdp hppmratglp sslasipggk
paysfhvsad gqmqpvpfph dalagpgipr harqintlsh
gevvcavtis nptrhvytgg kgcvkiwdis gpgskspisq
ldclnrdnyi rsckllpdgr tlivggeast ltiwdlaspt
prikaeltss apacyalais pdakvcfscc sdgniavwdl
hnqtivrqfq ghtdgascid ishdgtklwt ggldntvrsw
dlregrqlqq hdftsgifsl gycptgewla vgmessnvev
lhhtkpdkyq lhlhescvls lkfaycgkwf vstgkdnlln
awrtpygasi fqskesssvl scdisaddky ivtgsgdkka
tvyeviy
Human TLE4 protein
SEQ ID NO: 4
mirdlskmyp qtrhpaphqp aqpfkftise scdrikeefq
flqaqyhslk leceklasek temqrhyvmy yemsyglnie
mhkqaeivkr lnaicaqvip flsqehqqqv vqaverakqv
tmaelnaiig qqlqaqhlsh ghglpvpltp hpsglqppai
ppigssagll alssalggqs hlpikdekkh hdndhqrvsp
sasfrgaekh rnsadysses kkqkteekei aarydsdgek
sddnlvvdvs nedpssprgs pahsprengl dktrllkkda
pispasiass sstpsskske lslneksttp vsksntptpr
tdaptpgsns tpglrpvpgk ppgvdplass lrtpmavpcp
yptpfgivph agmngeltsp gaayaglhni spqmsaaaaa
aaaaaaygrs pvvgfdphhh mrvpaippnl tgipggkpay
sfhvsadgqm qpvpfppdal igpgiprhar qintlnhgev
vcavtisnpt rhvytggkgc vkvwdishpg nkspvsqldc
lnrdnyirsc rllpdgrtli vggeastlsi wdlaaptpri
kaeltssapa cyalaispds kvcfsccsdg niavwdlhnq
tivrqfqght dgascidisn dgtklwtggl dntvrswdlr
egrqlqqhdf tsqifslgyc ptgewlavgm ensnvevlhv
tkpdkyqlhl hescvlslkf ahcgkwfvst gkdnllnawr
tpygasifqs kesssvlscd isvddkyivt gsgdkkatvy
eviy
Human ILE5 protein
SEQ ID NO: 5
mchkngfpqe ggitaaflqk rklrlsknhr parakvtehv
rgtrpgrata gpaastraag slffdrwgnr gpagcrgssh
lpqqlkftts dscdrikdef qllqaqyhsl klecdklase
ksemqrhyvm yyemsyglni emhkqaeivk ringicaqvl
pyisqehqqq vlgaierakq vtapelnsii rqqlqahqls
qlqalalplt plpvglqpps lpaysagtgl lslsalgsqa
hlskedkngh dgdthqeddg eksd
Human TLE1 coding sequence
SEQ ID NO: 6
atgttcccgcagagccggcacccgacgccgcaccaggctgcagg
ccagcccttcaagttcactatcccggagtccctggaccggatta
aagaggaattccagttcctgcaggcgcagtatcacagccttaaa
ttggaatgtgagaaactggcaagtgaaaagacagaaatgcagag
gcactatgtgatgtattatgaaatgtcatatggattaaacattg
aaatgcacaaacagactgaaatcgccaagagattgaatacgatt
tgtgcacaagtcatcccatttctgtctcaggaacatcaacaaca
ggtggcccaggctgttgaacgtgccaaacaggtgaccatggcag
agttgaatgccatcatcgggcagcagcagttgcaagctcagcat
ctttctcatggccacggacccccagttccccttacgcctcaccc
ttcgggacttcagcctcctggaatcccgcccctcgggggcagtg
ccggccttcttgcgctgtctagtgctctgagtgggcagtctcac
ttggcaataaaagatgacaagaagcaccacgatgcagagcacca
cagagacagagagccgggcacaagtaattccctcctggtcccag
acagtctaagaggcacagataaacgcagaaatggacctgaattt
tccaatgacatcaagaaaaggaaggtggatgataaggactccag
ccactatgacagtgatggtgacaaaagcgatgacaacttagttg
tggatgtgtctaatgaggacccttcttctccgcgagcaagccct
gcccactcgccccgggaaaatggaatcgacaaaaatcgcctgct
aaagaaggatgcttctagcagtccagcttccacggcctcctcgg
caagttccacttctttgaaatccaaagaaatgagcttgcatgaa
aaagccagcacgcctgttctgaaatccagcacaccaacgcctcg
gagcgacatgccaacgccgggcaccagcgccactccaggcctcc
gtccaggtctcggcaagcctccagccatagaccccctcgttaac
caagcggcagctggcttgaggacacccctggcagtgcccggccc
atatcctgctccttttgggatggtcccccacgctggcatgaacg
gcgagctgaccagcccaggcgctgcctacgccagtttacacaac
atgtcgccccagatgagcgccgcagccgccgcggccgccgtggt
ggcctacgggcgctcccccatggtggggtttgatcctccccctc
acatgagagtacctaccattcctccaaacctggcaggaatccct
ggggggaaacctgcatactccttccacgttactgcagacggtca
gatgcagcctgtcccttttccccccgacgccctcatcggacccg
gaatcccccggcatgctcgccagatcaacaccctcaaccacggg
gaggtggtgtgcgctgtgaccatcagcaaccccacgagacacgt
gtacacaggcgggaagggctgcgtcaaggtctgggacatcagcc
accctggcaataagagccctgtctcccagctcgactgtctgaac
agagacaattatatccgttcctgtaaattgctacccgatggctg
cactctcatagtgggaggggaagccagtactttgtccatttggg
acctggcggctccaaccccgcgcatcaaggcggagctgacgtcc
tcggcccccgcctgctacgccctggccatcagccccgattccaa
ggtctgcttctcatgctgcagcgacggcaacatcgctgtgtggg
atctgcacaaccagacactagtgaggcaattccagggccacaca
gacggagccagctgtattgacatttctaatgatggcaccaagct
ctggacgggtggtttggacaacacagtcaggtcctgggacctgc
gcgaggggcggcagctgcagcagcacgacttcacctcccagatc
ttctccctggggtactgccccaccggggagtggctggcagtggg
catggagagcagcaatgtggaggtgctgcacgtgaacaagcctg
acaagtaccagctgcacctgcatgagagctgcgtgctgtccctg
aaatttgcttactgtggtaaatggtttgtgagtactggaaaaga
taacctcctcaatgcttggcggaccccctatggagccagcatat
tccagtccaaagagtcctcgtcagtgcttagctgtgacatctct
gtggatgataagtacatagtcactggctcgggggacaagaaggc
tacagtctatgaagtcatctactga
Human TLE2 coding sequence
SEQ ID NO: 7
atgtacccccagggaaggcacccgaccccgctccagtccggcca
gcccttcaagttctcgatcttggagatctgcgaccgcatcaaag
aagaattccagtttcttcaggctcaataccacagcctcaagcta
gaatgtgagaagctggccagcgagaagacggaaatgcagcgaca
ttatgtcatgtattatgagatgtcgtacgggctcaacattgaaa
tgcataagcaggcggagattgtgaagcgtctgagcggtatctgc
gctcagattatccccttcctgacccaggagcatcagcagcaggt
gctccaggccgtagaacgcgccaagcaggtcaccgtgggggagc
tgaacagcctcatcgggcagcagctccagccgctgtcccaccac
gcaccccctgtgcccctcaccccccgcccagccgggctggtggg
cggcagtgctacggggctgcttgctctgtctggagccctggagc
cctggctgcccaggctcagctggcggcggctgtcaaggaggacc
gtgcgggcgtggaggccgaggggtccagagtggagagagccccg
agcaggagtgcatctccctcgccccctgagagtctcgtggagga
ggagcgaccgagtggccctggtggtggcgggaagcagagagcag
atgagaaggagccatcaggaccttatgaaagcgacgaagacaag
agtgattacaatctggtggtggacgaggaccaaccctcagagcc
ccccagcccggctaccaccccctgcggaaaggtacccatctgca
ttcctgcccgtcgggacctggtggacagtccagcctccttggcc
tctagccttggctcaccgctgcctagagccaaggagctcatcct
gaatgaccttcccgccagcactcctgcctccaaatcctgtgact
cctccccgccccaggacgcttccacccccgggcccagctcggcc
agtcacctctgccagcttgctgccaagccagcaccttccacgga
cagcgtcgccctgaggagccccctgactctgtccagtcccttca
ccacgtccttcagcctgggctcccacagcactctcaacggagac
ctctccgtgcccagctcctacgtcagcctccacctgtcccccca
ggtcagcagctctgtggtgtacggacgctcccccgtgatggcat
ttgagtctcatccccatctccgagggtcatccgtctcttcctcc
ctacccagcatccctgggggaaagccggcctactccttccacgt
gtctgcggacgggcagatgcagccggttcccttcccctcggatg
cactggtaggcgcgggcatcccggcggcacgcccggcagctgca
cacgctggcccatggcgaggtggtctgcgcggtcaccatcagcg
gctccacacagcatgtgtacacgggcggcaagggctgtgtgaag
gtgtgggacgtgggccagcctggggccaagacgcccgtggccca
gctcgactgcctgaaccgagacaactacattcgttcctgcaagt
tgctgccggatggccggagtctgatcgtgggcggtgaggccagc
accttgtccatttgggacctggcggcgcccaccccccgtatcaa
ggccgagctgacttcctcagccccagcctgctacgccctggccg
tcagccccgacgccaaggtttgcttctcctgctgcagcgatggc
aacattgtggtctgggacctgcagaatcagactatggtcaggca
gttccagggccacacggacggcgccagctgcattgatatttccg
attacggcactcggctctggacagggggcctggacaacacggtg
cgctgctgggacctgcggagggccggccagctgcagcagcatga
cttcagctcccagattttctccctgggccactgccctaaccagg
actggctggcggtcggaatggagagtagcaacgtggagatcctg
cacgtccgcaagccggagaaataccagctgcacctccacgagag
ctgcgtgctgtccctgaagtttgcctcctgcggacggtggtttg
tgagcaccgggaaggacaacctgctcaacgcctggaggacgccg
taggggccagcattttccagtccaaggagtcgtcctcagtcctg
agttgtgacatctccagaaataacaaatacatcgtgacaggctc
gggggacaagaaggccaccgtgtatgaggtggtctactga
Human TLE3 coding sequence
SEQ ID NO: 8
atgtatccgcagggcagacatccggctcccatcaacccgggcag
ccgggatttaaattcacggtggctgagtcttgtgacaggatcaa
agacgaattccagttcctgcaagctcagtatcacagcctcaaag
tggagtacgacaagctggcaaacgagaagacggagatgcagcgc
cattatgtgatgtactatgagatgtcctatggcttgaacattga
aatgcacaagcagacagagattgcgaagagactgaacacaattt
tagcacagatcatgcctttcctgtcacaagagcaccagcagcag
gtggcgcaggcagtggagcgcgccaagcaggtcaccatgacgga
gctgaacgccatcatcgggcagcagcagctccaggcgcagcacc
tctcccatgccacacacggccccccggtccagttgccaccccac
ccgtcaggtctccagcctccaggaatccccccagtgacagggag
acagctccgggctgctggcactgggcgccctgggcagccaggcc
catctgacggtgaaggatgagaagaaccaccatgaactcgatca
cagagagagagaatccagtgcgaataactctgtgtcaccctcgg
aaagcctccgggccagtgagaagcaccggggctctgcggactac
agcatggaagccaagaagcggaaggtggaggagaaggacagctt
gagccgatacgacagtgatggagacaagagtgatgatctggtgg
tggatgtttccaatgaggaccccgcaacgccccgggtcagcccg
gcacactcccctcctgaaaatgggctggacaaggcccgtagcct
gaaaaaagatgcccccaccagccctgcctcggtggcctcttcca
gtagcacaccttcctccaagaccaaagaccttggtcataacgac
aaatcctccacccctgggctcaagtccaacacaccaaccccaag
gaacgacgccccaactccaggcaccagcacgaccccagggctca
ggtcgatgccgggtaaacctccgggcatggacccgataggtata
atggcctcggctctgcgcacgcccatctccatcaccagctccta
tgcggcgcccttcgccatgatgagccaccatgagatgaacggct
ccctcaccagtcctggcgcctacgccggcctccacaacatccca
ccccagatgagcgccgccgccgctgctgcagccgctgcctatgg
ccgatcgccaatggtgagctttggagctgttggttttgaccctc
accccccgatgcgggccacaggcctcccctcaagcctggcctcc
attcctggaggaaaaccagcgtactcattccatgtgagtgctga
tgggcagatgcagcccgtgcccttcccccacgacgccctggcag
gccccggcatcccgaggcacgcccggcagatcaacacactcagc
cacgggggggtggtgtgtgccgtgaccatcagcaaccccagcag
gcacgtctacacaggtggcaagggctgcgtgaagatctgggaca
tcagccagccaggcagcaagagccccatctcccagctggactgc
ctgaacagggacaattacatgcgctcctgcaagctgcaccctga
tgggcgcacgctcatcgtgggcggcgagggcagcacgctcacca
tctgggacctggcctcgcccacgccccgcatcaaggccgagctg
acgtcctcggctcccgcctgttatgccctggccattagccctga
cgccaaagtctgcttctcctgctgcagcgatgggaacattgctg
tctgggacctgcacaaccagaccctggtcaggcagttccagggc
cacacagatggggccagctgcatagacatctcccatgatggcac
caaactgtggacagggggcctggacaacacggtgcgctcctggg
acctgcgggagggccgacagctacagcagcatgacttcacttcc
cagatcttctcgctgggctactgccccactggggagtggctggc
tgtgggcatggagagcagcaacgtggaggtgctgcaccacacca
agcctcacaagtaccagctgcacctgcacgagagctgcgtgctc
tccctcaagttcgcctactgcggcaagtggttcgtgagcactgg
gaaagataaccttctcaacgcctggaggacgccttatggagcca
gcatatcccagtctaaagaatcctcgtctgtcttgagttgtgac
atttcagcggatgacaaatacattgtaacaggctctggtgacaa
gaaggccacagtttatgaggtcatctactaa
Human TLE4 coding sequence
SEQ ID NO: 9
atgattcgcgacctgagcaagatgtacccgcagaccagacaccc
ggcaccgcatcagcctgctcaaccctttaaatttacaatttccg
aatcctgtgatcggattaaggaagagtttcagtttttacaggct
caataccacagtctgaagctggaatgtgagaaactcgccagtga
gaagacagagatgcagcggcattatgtcatgtattatgaaatgt
cctatgggttgaatatagaaatgcacaagcaggcagagattgtc
aagaggctgaatgctatctgtgcacaagtcattcctttcctgtc
ccaagagcaccagcaacaagtggtgcaggctgtggaacgggcca
agcaggtgaccatggcagaactgaacgccatcattgggcaacaa
ctccaggcccagcatttatcacatggacatggtctccccgtacc
tctgactccacacccttcagggctccagccccctgccattccac
ccatcggtagcagtgccgggcttctggccctctccagtgctcta
ggaggtcagtcccatcttccaattaaagatgagaagaagcacca
tgacaatgataccaaagagacagagactccatcaagagctcttc
agtatccccatcagccagtttccgaggtgctgagaagcacagaa
actccgcagactactactcctcagagagcaaaaagcagaaaact
gaagaaaaggaaattgcagctcgttatgacagcgatggtgagaa
aagtgatgacaacttggtggttgacgtttccaatgaggatccat
cttcccctcgagggagcccagcacattcccccagagagaatggc
ctagacaagacacgcctgctcaagaaagatgccccgattagtcc
agcctctattgcatcttccagcagtactccctcctccaaatcca
aagaacttagccttaatgaaaaatctactactcccgtctcaaag
tccaatacccctactccacgaactgatgcgcccaccccaggcag
taactctactcccggattgaggcctgtacctggaaaaccaccag
gagttgaccctttggcctcaagcctaaggaccccaatggcagta
ccttgtccatatccaactccatttgggattgtgccccatgctgg
aatgaacggagagctgaccagccccggagcggcctacgctgggc
tccacaacatctcccctcagatgagcgcagctgctgccgccgcc
gctgctgctgctgcctatgggagatcaccagtggtgggatttga
tccacaccatacacatgcgtgtgccagcaatacctccaaacctg
acaggcattccaggaggaaaaccagcatactccttccatgttag
cgcagatggtcagatgcagcctgtcccttttccacccgacgccc
tcatcggacctggaatcccccggcatgctcgccagatcaacacc
ctcaaccacggggaggtggtgtgcgcggtgaccatcagcaaccc
cacgagacacgtgtacacgggtgggaagggctgcgtcaaggtct
gggacatcagccacccaggcaataagagtcctgtctcccagctc
gactgtctgaacagggataactacatccgttcctgcagattgct
ccctgatggtcgcaccctaattgttggaggggaagccatacttt
gtccatttgggacctggcggctccaaccccacgcatcaaggcag
agctgacatcctcggcccccgcctgctatgccctggccatcagc
cccgattccaaggtctgcttctcatgctgcagcgacggcaacat
cgctgtgtgggatctgcacaaccagaccttggtgaggcaattcc
agggccacacagatggagccagctgtattgacatttctaatgat
ggcaccaagctctggacaggtggtttggacaacacggtcaggtc
ctgggacctgcgcgaggggcggcagctgcagcagcacgacttca
cctcccagatcttttctctgggctactgcccaactggagagtgg
cttgcagtggggatggagaacagcaatgtggaagttttgcatgt
caccaagccagacaaataccaactacatcttcatgagagctgtg
tgctgtcgctcaagtttgcccattgtggcaaatggtttgtaagc
actggaaaggacaaccttctgaatgcctggagaacaccttatgg
ggccagtatattccagtccaaagaatcctcatcggtgcttagct
gtgacatctccgtggacgacaaatacattgtcactggctctggg
gataagaaggccacagtttatgaagttatttattaa
Human TLE5 coding sequence
SEQ ID NO: 10
atgtgtcacaagaatggcttccctcaggaaggcggcattaccgc
tgcctttctgcagaaaaggaaactaaggctcagcaagaaccacc
gcccagccagagccaaggtcacagagcacgtccgtggcacgcgt
ccaggtcgtgccacagcagggccggcggcttcgacgcgggcagc
cggtcccttttctttgacagatggggaaaccgaggcccggccgg
ctgccggggctcctcgcacctaccccagcaactcaaattcacca
cctcggactcctgcgaccgcatcaaagacgaatttcagctactg
caagctcagtaccacagcctcaagctcgaatgtgacaagttggc
cagtgagaagtcagagatgcagcgtcactatgtgatgtactacg
agatgtcctacggcttgaacatcgagatgcacaaacaggctgag
atcgtcaaaaggctgaacgggatttgtgcccaggtcctgcccta
cctctcccaagagcaccagcagcaggtcttgggagccattgaga
gggccaagcaggtcaccgctcccgagctgaactctatcatccga
cagcagctccaagcccaccagctgtcccagctgcaggccctggc
cctgcccttgaccccactacccgtggggctgcagccgccttcgc
tgccggcggtcagcgcaggcaccggcctcctctcgctgtccgcg
ctgggttcccaggcccacctctccaaggaagacaagaacgggca
cgatggtgacacccaccaggaggatgatggcgagaagtcggatt
ag
Mouse Grg1 protein
SEQ ID NO: 11
mfpqsrhptp hqaagqpfkf tipesldrik eefqflqaqy
hslklecekl asektemqrh yvmyyemsyg lniemhkqte
iakrintica qvipflsqeh qqqvaqaver akqvtmaeln
aiigvrglpg lpptqqq1qa qhlshghgpp vpltphpsgl
qppgipplgg sasllalssa lsgqshlaik ddkkhhdaer
hrdrepgtsn sllvpdslrg tdkrrngpef ssdikkrkvd
dkdnydsdgd ksddnlvvdv snedpsspha spthspreng
idknrllkkd asgspastas sgsssslksk evslhekant
pvlksstptp rsdmptpgts atpglrpglg kppameplvn
qaaaglrtpl avpgpypapf gmvphagmng eltspgaaya
glhsmspqms aaaaaaaaav vaygrspmvg fdppphmrvp
sippnlagip ggkpaysfhv tadgqmqpvp fppdaligpg
iprharqint lnhgevvcav tisnptrhvy tggkgcvkvw
dishpgnksp vsqldclnrd nyirsckllp dgctlivgge
astlsiwdla aptprikael tssapacyal aispdskvcf
sccsdgniav wdlhnqtlvr qfqghtdgas cidisndgtk
lwtggldntv rswdlregrq lqqhdftsqi fslgycptge
wlavgmessn vevlhvnkpd kyqlhlhesc vlslkfaycg
kwfvstgkdn llnawrtpyg asifgskess svlscdisvd
dkyivtgsgd kkatvyeviy
Mouse Grg2 protein
SEQ ID NO: 12
mypqgrhptp lqsgqpfkfs vleicdrike efqflgagyh
slklecekla sektemqrhy vmaaphqcpq ggtsyphwpr
lsplqyyems yglniemhkq aeivkrlsai caqmvpfltq
ehqqqvlqav drakqvtvge 1nsiagqqnq lqp1shappv
pltprpaglv gagatgllal sgalaaqaql vaavkedrvg
vdaegsrvdr aasrssspsp peslveedhp ssrggsgkqq
raedkdlsgp ydseedksdy nlvvdedqps eppspvttpc
gkaplcipar rdltdspasl asslgsplpr skdialndlp
tgtpasrscg tsppqdsstp gpssashlcq laagpaaptd
sialrspltl sspftssfsl gshstlngdl smpgsyvglh
lspgvsssvv ygrsplqmaf eshphlrgss vslpgipvak
paysfhvsad ggmgpvpfps dalvgtgipr harqlhtlah
gevvcavtis sstqhvytgg kgcvkvwdvg qpgsktpvaq
ldclnrdnyi rsckllpdgq slivggeast lsiwdlaapt
prikaeltss apacyalays pdakvcfscc sdgnivvwdl
qnqamvrqfq ghtdgascid isdygtrlwt ggldntvrcw
dlregrqlqq hdfssqifsl ghcpnqdwla vgmesshvev
lhvrkpekyq lrlhescvls lkfascgrwf vstgkdnlln
awrtpygasi fqskesssvl scdisrnnky ivtgsgdkka
tvyevvy
Mouse Grg3 protein
SEQ ID NO: 13
mypqgrhpap hqpgqpgfkf tvaescdrik defqflqaqy
hslkveydkl anektemqrh yvmyyemsyg lniemhkqte
iakrintila gimpflsgeh qqqvaqaver akqvtmteln
aiigvrglpn lpltqqqlqa qhlshathgp pvqlpphpsg
lqppgippvt gsssgllalg algsqahlav kdeknhheld
hreresstnn syspseslra sekhrgsady smeakkrkae
ekdslsryds dgdksddlvv dvsnedpatp rvspahsppe
ngldkarglk kdaptspasv asssstpssk tkdlghndks
stpglksntp tprndaptpg tsttpglrsm pgkppgmdpi
gimasalrtp itltssypap fammshhemn gsltspsaya
glhnipsqms aaaaaaaaay grspmvsfga vgfdphppmr
atglpsslas ipggkpaysf hvsadgqmqp vpfphdalag
pgiprharqi ntlshgevvc avtisnptrh vytggkgcvk
iwdisqpgsk spisgldcln rdnyirsckl lpdgrtlivg
geastltiwd lasptprika eltssapacy alaispdakv
cfsccsdgni avwdlhnqtl vrqfqghtdg ascidishdg
tklwtggldn tvrswdlreg rqlqqhdfts qifslgycpt
gewlavgmes snvevlhhtk pdkyqlhlhe scvlslkfay
cgkwfvstgk dnllnawrtp ygasifqske sssvlscdis
addkyivtgs gdkkatvyev iy
Mouse Grg4 protein
SEQ ID NO: 14
mirdlskmyp qtrhpaphqp aqpfkftise scdrikeefq
flqaqyhslk leceklasek temqrhyvmy yemsyglnie
mhkqaeivkr lnaicaqvip flsgehqqqy vqaverakqv
tmaelnaiig qqlqaqhlsh ghglpvpltp hpsglgppai
ppigssagll alssalggqs h1pikdekkh hdndhqrdrd
siksssysps asfrgsekhr nstdyssesk kqkteekeia
arydsdgeks ddnlvvdvsn edpssprgsp ahsprengld
ktrllkkdap ispasvasss stpsskskel slneksttpv
sksntptprt daptpgsnst pglrpvpgkp pgvdplassl
rtpmavpcpy ptpfgivpha gmngeltspg aayaglhnis
pqmsaaaaaa aaaaaygrsp vvgfdphhhm rvpaippnit
gipggkpays fhvsadgqmq pvpfppdali gpgiprharq
intlnhgevv cavtisnptr hvytggkgcv kvwdishpgn
kspvsqldcl nrdnyirscr llpdgrtliv ggeastlsiw
dlaaptprik aeltssapac yalaispdsk vcfsccsdgn
iavwdlhnqt lvrqfqghtd gascidisnd gtklwtggld
ntvrswdlre grqlqqhdft sqifslgycp tgewlavgme
nsnvevlhvt kpdkyqlhlh escvlslkfa hcgkwfvstg
kdnllnawrt pygasifgsk esssvlscdi svddkyivtg
sgdkkatvye viy
Mouse Grg5 protein
SEQ ID NO: 15
mmfpqsrhsg sshipqqlkf ttsdscdrik defqllqaqy
hslklecdkl aseksemqrh yvmyyemsyg lniemhkqae
ivkringica qvlpylsgeh qqqvlgaier akqvtapeln
siirqqlqah qlsqlqalal pltplpvglq ppslpaysag
tgllslsalg sgthlskedk nghdgdthqe ddgeksd
Human MDM2 protein
SEQ ID NO: 16
mvrsrqmcnt nmsvptdgav ttsqipaseq etivrpkpll
lkllksvgaq kdtytmkevl fyiggyimtk rlydekqqhi
vycsndllgd lfgvpsfsvk ehrkiytmiy rnlvvvnqqe
ssdsgtsyse nrchleggsd qkdlvgelge ekpssshlvs
rpstssrrra iseteensde lsgerqrkrh ksdsislsfd
eslalcvire iccersssse stgtpsnpdl dagvsehsgd
widgdsysdq fsvefevesl dsedyslsee gqelsdedde
vyqvtvyqag esdtdsfeed peisladywk ctscnemnpp
lpshcnrcwa lrenwlpedk gkdkgeisek aklenstqae
egfdvpdckk tivndsresc veenddkitq asgsgesedy
sqpstsssii yssqedvkef ereetqdkee svesslpina
iepcvicqgr pkngcivhgk tghlmacftc akklkkrnkp
cpvcrqpiqm ivltyfp
Human MDM2 coding sequence
SEQ ID NO: 17
atggtgaggagcaggcaaatgtgcaataccaacatgtctgtacc
tactgatggtgctgtaaccacctcacagattccagcttcggaac
aagagaccctggttagaccaaagccattgcttttgaagttatta
aagtctgttggtgcacaaaaagacacttatactatgaaagaggt
tcttttttatottggccagtatattatgactaaacgattatatg
atgagaagcaacaacatattgtatattgttcaaatgatcttcta
ggagatttgtttggcgtgccaagcttctctgtgaaagagcacag
gaaaatatataccatgatctacaggaacttggtagtagtcaatc
agcaggaatcatcggactcaggtacatctgtgagtgagaacagg
tgtcaccttgaaggtgggagtgatcaaaaggaccttgtacaaga
gcttcaggaagagaaaccttcatcttcacatttggtttctagac
catctacctcatctagaaggagagcaattagtgagacagaagaa
aattcagatgaattatctggtgaacgacaaagaaaacgccacaa
atctgatagtatttccetttcctttgatgaaagcctggctctgt
gtgtaataagggagatatgttgtgaaagaagcagtagcagtgaa
tctacagggacgccatcgaatccggatcttgatgctggtgtaag
tgaacattcaggtgattggttggatcaggattcagtttcagatc
agtttagtgtagaatttgaagttgaatctctcgactcagaagat
tatagccttagtgaagaaggacaagaactctcagatgaagatga
tgaggtatatcaagttactgtgtatcaggcaggggagagtgata
cagattcatttgaagaagatcctgaaatttecttagctgactat
tggaaatgcacttcatgcaatgaaatgaatcccccccttccatc
acattgcaacagatgttgggcccttcgtgagaattggcttcctg
aagataaagggaaagataaaggggaaatctctgagaaagccaaa
ctggaaaactcaacacaagctgaagagggctttgatgttcctga
ttgtaaaaaaactatagtgaatgattccagagagtcatgtgttg
aggaaaatgatgataaaattacacaagcttcacaatcacaagaa
agtgaagactattctcagccatcaacttctagtagcattattta
tagcagccaagaagatgtgaaagagtttgaaagggaagaaaccc
aagacaaagaagagagtgtggaatctagtttgccccttaatgcc
attgaaccttgtgtgatttgtcaaggtcgacctaaaaatggttg
cattgtccatggcaaaacaggacatcttatggcctgctttacat
gtgcaaagaagctaaagaaaaggaataagccctgcccagtatgt
agacaaccaattcaaatgattgtgctaacttatttcccctag
Mouse Grg1 coding sequence
SEQ ID NO: 18
atgttcccgcagagccggcacccaacsccgcaccaagctgcagg
ccagccctttaagttcactatcccggagtctctggaccggatta
aagaggaattccagttcctgcaggcgcagtatcacagtcttaaa
ttggagtgtgagaaactggcaagtgaaaagacagaaatgcagag
gcactacgtgatgtattatgaaatgtcatatggattaaacattg
aaatgcacaaacagactgaaatcgccaagcgcttgaacaccatt
tgtgcccaagacatcccatttctgtctcaggaacatcaacaaca
ggtggcccaggctgtggaacgtgccaaacaggtgaccatggcag
agttgaatgccatcatcgggcagcagcagttgcaagctcagcat
ctctcccatggccatgacccccagtacctctcacgcctcaccct
tcaggacttcagcctcctggaatcccgcccctcgggggcagtgc
cggccttcttgcgctgtctagtgctctgagtgggcagtctcact
tggcaataaaagatgacaagaagcaccatgatgcagagcgccac
agagacagagagcctggcacgagtggttccctcttggtcccaga
cagcctaagaggcacagataagcgcagaaatggtccagagtttt
ccagtgacatcaaaaaaaggaaggtggatgataaggataactat
gacagtgatggggacaagagtgatgacaacttagttgtggatgt
gtctaacgaggacccttcttctccacatgcaagccccacacact
caccccgggaaaacggattgacaaaaaccgtctgctgaagaaag
atgcttcaggtagcccggcatccacagcctcctctggaagttcc
tcttccctgaaatccaaagaagtgagcctgcatgaaaaagccaa
cactcctgttctgaaatccagcacaccgacgcctcggagcgaca
tgccaaccccaggcaccagcgctactccaggcctccgtccaggt
cttggcaagcctccagccatggaaccccttgtcaaccaagcagc
agctggcctgaggacgcccctggcagtgcctggcccataccctg
ccccctttggcatggtgccccatgcgggcatgaacggagagctg
accagccctggtgctgcctatgcaggtctacacagcatgtctcc
acagatgagcgctgcagctgctgcagctgctgctgctgtggtgg
cctatgggcgctccccaatggttggttttcatggtggtggtcac
atgagagtaccttctatcccccccaacctggcaggaatacctgg
agggaaaccagcatactccttccacgttactgctgatggccaaa
tgcagcctgttccttttccccctatgcccctcattggacctgga
ttccccgacatgctcggcagatcaacaccctcaaccacggggag
gtggtgtgcgcagtgaccatcagcaaccccacaaggcacgtgta
cacaggtggcaagggctgcgttaaggtgtgggacatcagccacc
ccggcaacaagagccccgtctctcagctggattgtctgaataga
gataactacatccgaacctgtaaattgctacctgatgacgtact
ctcatagtgggaggggaagccagtactttgtccatttgggacct
ggcggctccaaccccgcgcatcaaggcggagctgacgtcctcgg
cccccgcctgctacgccctggccatcagccccgactccaaggtc
tgcttctcatgctgcagtgacggcaacatcgcagtgtgggacct
gcacaaccagaccctggtgaggcaattccagggccacacagacg
gagccagctgtattgacatttctaatgatggcaccaagctctgg
acaggcggtttagacaacactgtgaggtcctgggacctgagaga
agggcggcagctgcagcagcatgacttcacttcacagatcttct
ctttgggatactgcccaactggggagtggcttgctgtgggcatg
gaaagcagcaacgtggaagttctgcatgtgaacaagcctgacaa
gtaccagctgcacctccacgagagctgcgtgctgtccctaaagt
ttgcttattgtggcaaatggtttgtgagtactggaaaagataac
ctcctcaatgcttggcggaccccctatggagccagcatattcca
gtccaaagagtcctcgtcagtgcttagctgtgacatctctgtgg
atgataagtacatagtcactggctcgggggacaagaaggctaca
gtctatgaagtcatctactga
Mouse Grg2 coding sequence
SEQ ID NO: 19
atgtaccctcagggaaggcacccgaccccgctgcagtctggcca
gcctttcaagttctcagtactggaaatctgtgaccggatcaaag
aggaattccagtttcttcaagctcagtaccacagcctcaagcta
gaatgtgagaagctggccagcgagaagacagaaatgcaaaggca
ttatgtgatggctgcaccccatcagtgtccccagggtggcacca
gctatccacactggccaagactgtctcctttgcagtactacgag
atgtcctacggactcaacattgagatgcataaacaggctgagat
tgtgaaacgcctcagtgcgatctgtgcccagatggtcccgttcc
tcactcaggagcatcagcagcaggtgctccaggctgtggaccga
gccaagcaggtgaccgtgggggaactgaacagcctcctggggca
gcagaatcagctccagccgctgtcccacgcaccccccgtgcctc
tcaccccgcgcccagccggcctggtgggtgccggggccactggg
ctgctggccctatctggggcactggctgcgcaggcccagctggt
ggctgccgtaaaggaagaccgtgtgggtgtggacgccgaggggt
ccagagtggacagagctgccagcaggagttcttccccgtctccc
cctgagagtctggtggaagaggaccatcccagcagccgaggcgg
cagtgggaaacagcagagagctgaagacaaggatctgtcagggc
cttatgacagtgaggaagacaagagtgactataacctggtagtg
gatgaggaccaaccgtcagagccccccagccctgtgaccacccc
ttgtgggaaggcgcccctctgcattcctgcccgcagggacctca
cagacagtccagcctccttggcctccagtttgggctcaccactc
cccagaagcaaagacatagccctgaacgatcttcccacaggcac
tcctgcctccaggtcatgtggtacctctccaccccaggactcgt
ccaccccggggcccagctcagccagtcacctctgccagctggcg
gctcagccggcagcacccacagacagcatcgaccctgaggagtc
ccctgaccttgtccagccctttcacctcatccttcagcctgggc
tcccacagcaccctcaatggggacctctccatgcctggctccta
tgtcggcctccacctgtccccccaggtcagcagctctgtcgtgt
atggacgctcacctctgcagatggcatttgaatcgcacccccat
ctccgaggctcgtctgtctccttgcctggcatccctgtggctaa
gccggcttactccttccacgtgtctgcggatggccagatgcagc
ctgtgcccttcccgtctgatgctctggtaggcacaggcatccct
cgccacgcaaggcagctacacacgctggcccacggtgaggtggt
gtgtgccgtcaccatcagcagctccacacagcacgtgtacacag
gcggcaaaggatgcgtgaaggtgtgggacgtgggccagccgggt
agcaagacccctgtggcacagctggattgcctgaaccgagacaa
ctacatccgctcctgcaagctgctgcccgacgggcagagcctga
ttgtaggtggcgaggccagtaccctgtccatttgggacctggca
gcacccacaccacggatcaaagcagagctgacttcgtctgcccc
ggcctgctatgctctggccgtcaagtccggacgccaaggtctgc
ttctcctgctgcagcgacggtaacatcgtggtctgggacctgca
gaaccaggccatggtcagacagttccagggccacacggacgggg
ccagctgcatcgacatatcagactacgggacccggctgtggact
gggggcctggacaacactgtgcgctgctgggacctgcgcgaggg
ccgccagcttcagcagcatgacttcagttcccagattttctcat
tgggccactgtcccaatcaggactggttggctgtggggatggag
agcagccacgtggaggtcctgcatgtgcgcaagcctgagaagta
ccagctccgcctccatgagagctgcgtgctgtcactcaagttcg
cttcctgtggacgctggtttgtgagcacaggcaaggacaacctg
ctcaatgcctggaggacaccctatggggccagcatttttcagtc
caaagaatcatcttctgtactgagctgcgacatctccaggaata
ataagtacatcgtgacaggctcaggggacaagaaggccactgtg
tatgaggtggtgtactga
Mouse Grg3 coding sequence
SEQ ID NO: 20
atgtatccgcaaggcagacatccggcaccccatcaacccgggca
gccgggatttaaattcactgtggccgagtcctgtgacaggatca
aagacgaattccagttcctgcaagctcagtatcacagcctcaaa
gtggagtatgacaagctggctaacgagaagacggagatgcagcg
ccattatgtgatgtactatgagatgtcctatggcttgaatattg
aaatgcacaagcagacagagattgcgaagagactgaacacaatc
ctagcccagatcatgccttttttgtcacaggagcatcagcagca
agtggcgcaggctgtggaacgcgccaagcaggtcaccatgacgg
agttgaacgccatcatcggggtacgtggactccccaatctgcct
ctcacccagcagcaactccaggcccagcacctctcccatgccac
gcatggtcccccggtccagctgccaccccacccgtcaggcctcc
agcctcctgggattcccccagtgacaggaagcagctctgggttg
ctggcacttggtgccctgggaagtcaagctcacttggcggtgaa
ggatgagaagaaccaccatgaactggatcacagagagagagagt
ccagcacgaacaatccgtgtcaccctctgaaagcctccgggcca
gtgagaagcaccggggctctgcagactacagcatggaagccaag
aagcggaaggcggaagagaaagacagcctcagcagatacgatag
cgatggggacaagagtgacgacctggtggtggatgtctctaatg
aggacccagcaacaccccgggtgagcccagcacactcccctcct
gaaaatgggctggacaaagcccgtggtctgaagaaagatgcccc
caccagcccagcctccgtggcttcctccagcagcacaccttcct
ccaagaccaaagaccttggtcataatgacaaatcttccacacct
gggctcaagtccaacacaccaacgccaagaaatgatgccccaac
tccaggcaccagcaccaccccggggctccggtcaatgccgggca
aacctccaggcatggacccgataggtataatggcctcggccctg
cgaacacccatcaccctcaccagctcctatccagcaccctttgc
catgatgagccaccacgagatgaatggctccctcaccagcccaa
gcgcctatgctggcctacacaacatcccatcccagatgagcgcc
gccgcagccgctgcagccgccgcctatggccgatcgccaatggt
gagctttgggagctgttggttttgaccctcaccccccaatgagg
gccacaggcctgccttccagtctcgcctccattcctggagggaa
accggcatatctccttccatgtgagtgctgatgggcagatgcaa
cctgtgcccttcccccatgatgcactagcaggccctggcattcc
caggcatgcccggcagatcaatacgctcagccatggagaggtgg
tatgtgctgtgaccatcagcaaccccacacgacacgtctacaca
ggcggcaagggctgtgtgaagatatgggacatcagccagccggg
cagcaagagtcccatctcccagctggactgcctgaacagggaca
actacatccgctcgtgcaagcttctccccgatgggcgcacgctc
attgtgggtggtgaggccagcacgctcaccatctgggacctggc
ctcacccacaccccgcatcaaggctgagctgacgtcctcggctc
cagcctgttatgccctggccatcagtggtgatgccaaagtctgt
ttttcctgctgcagcgacgggaacattgcggtttgggatctgca
caaccagaccctggtcaggcagttccagggccacacagatgggg
ccagctgtatagacatctctcatgatggcactaagctgtggacc
gggggcctggacaacaccgtgcgctcctgggacctacgtgaagg
acggcagttacagcaacacgatttcacctcccagatcttctccc
tgggttactgccccactggggagtggctggccgtgggcatggag
agcagcaatgtggaggtcctgcaccacactaagcccgacaaata
ccagctgcacctgcacgagagctgcgtgctgtccctcaagttcg
cctattgtggcaagtggtttgtgagcactgggaaagacaacctt
ctcaatgcctggaggacgccttatggagccagcatcttccagtc
aaaagaatcctcatctgtcttgagctgtgacatttcagcggatg
acaaatatattgtaacaggctctggtgacaagaaggccacagtt
tacgaggtcatctactga
Mouse Grg4 coding sequence
SEQ ID NO: 21
atgattcgcgacctgagcaagatgtacccgcagacgcgccaccc
ggcaccgcatcagcctgcaacccttcaaatttacaatttcagaa
tcctgtgatcggattaaggaagagtttcagtttttacaggctca
ataccacagtctgaagctggaatgtgagaagctcgccagcgaga
agacagagatgcagcggcattatgtcatgtattatgaaatgtcc
tatgggttgaacatagaaatgcacaagcaggcagagattgttaa
acgactaaatgctatctgtgcacaggtcattcctttcctgtccc
aagagcaccagcaacaagtggtgcaggctgtggaacgggccaag
caggtgaccatggcagaactgaacgccatcattgggcaacaact
ccaagctcagcatttatcacatggacatggtctgcctgtgcctc
tgacaccacacccttcagggcttcagcccccagccatcccacct
atcggtagcagtgcaggacttctggccctctccagtgcactagg
aggtcagtctcacctcccaattaaggatgagaagaagcaccatg
acaatgatcaccaaagagacagagactccatcaagagctcttcc
gtatccccatcagccagtttccggggttctgagaaacataggaa
ctctacagactattcctcagagagcaaaaagcagaaaacagaag
aaaaagaaattgcagctcgctatgacagtgatggtgagaagagt
gatgacaacttggtagttgatgtgtccaatgaggatccatcttc
cccccgaggaagcccagcacattccccaagggagaatggcctgg
acaagacacgactcctcaagaaagatgcccccatcagccccgct
tctgttgcatcttccagcagtactccttcctccaaatccaaaga
gcttagccttaatgaaaagtctactactcctgtttcaaagtcca
atacccctactccacgaactgatgcacctacccctggcagtaat
tctactcctggactgaggcctgtacctggaaagccaccaggtgt
ggaccccttagcatcaagcctgaggaccccgatggctgtacctt
gtccatatccaaccccctttgggatcgtgccccatgctgggatg
aacggagagctgaccagccccggagccgcctatgctgggctaca
caacatctccccccagatgagtgcagctgctgctgcagctgcgg
cagcagcagcctatggaagatcacctgtggtgggatttgatcca
caccatcacatgcgagtgccagcaatacctccaaatctaacagg
cattccaggaggaaaaccagcatactcctttcacgtcagtgccg
atggccagatgcagcctgtccctttcccccctgatgccctcatt
ggaccagggatcccccgacacgctcgacagatcaacaccctcaa
ccatggggaggtggtgtgtgcagtgaccatcagcaaccccacaa
ggcacgtgtacacaggtggcaagggctgtgttaaggtctgggac
atcagccaccctggcaacaagagcccagtctctcagctggactg
tctgaacagggataactacatccgttcctgcagattgctccctg
atggtcgcaccttaattgttggaggggaagccagcacactgtcc
atctgggacctggcagctccaactccacgcatcaaggcagagct
gacatcctcagcccctgcctgctatgctctggccatcagccccg
actccaaggtctgcttctcatgctgcagcgacggtaacagcgca
gtgtgggatctgcacaaccagactctggtgaggcaattccaggg
gcacacagatggagccagctgtattgacatttctaatgatggca
ccaagctctggacaggtggtttggacaacactgtgaggtcctgg
gacctgcgtgaagggcggcagctgcagcaacatgacttcacctc
tcagatcttttcattgggctattgcccaactggagagtggcttg
cagtggggatggagaatagcaatgtggaagtattgcatgtcacc
aaaccagacaaataccagttgcatcttcatgagagctgtgtgct
gtcactcaagtttgcccactgtggcaaatggtttgtaagcactg
gaaaggacaacttctgaatgcttggaggacgccttatggggcca
gcatattccagtccaaagaatcctcatcggtgcttagctgtgac
atctctgtggatgacaagtacattgtcactggctctggggacaa
gaaagctacggtttatgaagttatttattaa
Mouse Grg5 coding sequence
SEQ ID NO: 22
atgatgtttccgcaaagccggcactcgggctcctcccacctccc
tcagcagctcaagttcaccacctccgactcctgtgaccgcatca
aagatgagttccagctgctgcaagcgcagtatcacagcctgaag
ctggagtgcgacaagctggccagcgagaagtcagagatgcagag
gcattacgtcatgtactatgagatgtcctacggattgaacatcg
agatgcacaaacaggcggaaattgtgaagaggctgaatgggatt
tgcgcccaggttctgccctatttgtcacaggagcatcagcagca
ggtcctgggagccatcgagagagccaagcaggtcacggctcctg
agctgaactccatcatccgacagcagctccaggctcaccagctg
tcccagctgcaggcgctggccctgcccctgacaccgctgcctgt
cgggctccagccaccgtccctccctgcagtcagtgcaggcacag
gcctgctgtcactctctgctctgggttctcagacccacctctcc
aaggaggacaagaacggacacgatggggacacccaccaggagga
tgacggagagaagtcggattag

1. A method for diagnosing lung cancer in a mammal comprising:
providing a biological sample from said mammal;
providing a panel comprising one or more macromolecules, wherein each macromolecule is a biomarker or specifically binds to a biomarker that is measurable at a quantitatively different level in Grg1-expressing transgenic mice than in non-transgenic mice;
contacting the biological sample to the panel to determine the level of expression of said biomarker; [symbol] and
comparing the levels of said biomarker expressed in said biological sample to the levels of said biomarker in a reference sample;
wherein the presence of a quantitatively different level of expression of said biomarker in said mammalian biological sample compared to said reference sample is indicative of cancer.

2. The method of claim 1 wherein said reference sample is selected from the group consisting of a biological sample from a healthy mammal not diagnosed with cancer and not having increased risk factors for cancer; and a purified biomarker provided at a concentration level corresponding to the level measured in a biological sample from a healthy mammal not diagnosed with cancer and not having increased risk factors for cancer.

3. The method of claim 1 wherein said one or more macromolecule is selected from the group consisting of antibodies, nucleic acids, proteins and fragments thereof.

4. The method of claim 1 wherein said biomarker is selected from the group consisting of proteins, mRNA and antibodies.

5. The method of claim 4 wherein said biomarker is a protein selected from the group consisting of a Groucho-related protein, Mdm2, Ras, ErbB1, ErbB2 and CyclinD1/D2.

6. The method of claim 4 wherein said biomarker is an mRNA encoding a protein selected from the group consisting of a Groucho-related protein, Mdm2, Ras, ErbB1, ErbB2 and CyclinD1/D2.

7. The method of claim 4 wherein said biomarker is an autoantibody which specifically binds to a protein selected from the group consisting of a Groucho-related protein, Mdm2, Ras, ErbB1, ErbB2 and CyclinD1/D2.

8. The method of claim 1 wherein at least one of the said biomarkers comprises a Groucho-related protein.

9. The method of claim 1 wherein said panel comprises one or more antibody or fragment thereof, wherein said antibody specifically binds to a biomarker protein that is measurable at a quantitatively different level in Grg1-expressing transgenic mice than in non-transgenic mice

10. The method of claim 1 wherein said panel comprises one or more cDNA molecules or fragment thereof, wherein said cDNA molecules specifically bind to a biomarker mRNA or its complementary sequence, wherein said biomarker mRNA or its complementary sequence is measurable at a quantitatively different level in Grg1-expressing transgenic mice than in non-transgenic mice.

11. The method of claim 10 wherein said method further comprises isolating mRNA from said mammalian biological sample and said reference sample and quantitatively amplifying said mRNA and producing cDNA.

12. The method of claim 1 wherein said panel comprises one or more protein or fragment thereof that is a biomarker that is measurable at a quantitatively different level in Grg1-expressing transgenic mice than in non-transgenic mice.

13. The method of claim 1 wherein said biological sample is a fluid sample.

14. The method of claim 13 wherein said fluid sample is selected from the group consisting of blood, plasma and serum.

15. The method of claim 1 wherein said biological sample and said reference sample are detectably labeled.

16.-55. (canceled)

56. A diagnostic kit comprising one or more biomarker that is at a quantitatively different level in Grg1-expressing transgenic mice than in non-transgenic mice.

57. The kit of claim 56 wherein said one or more biomarker is selected from the group consisting of a Groucho-related protein, Mdm2, Ras, ErbB1, ErbB2 and CyclinD1/D2.

58. The kit of claim 56 wherein at least one biomarker comprises a Groucho-related protein.

59. The kit of claim 56 wherein said one or more biomarker is provided as a panel of biomarkers bound to a solid support.

60.-75. (canceled)
